(19) United States
(12) Patent Application Publication
(10) Pub. No.: US 2013/0274134 A1
(43) Pub. Date: Oct. 17, 2013

(54) Analytical Methods and Arrays for Use in the Same

(71) Applicants: Malin Lindstedt, Bunkeflostrand (SE); Carl Arne Krister Borrebaeck, Lund (SE); Henrik Johansson, Malmo (SE); Ann-Sofie Albrekt, Teckomatorp (SE)

(72) Inventors: Malin Lindstedt, Bunkeflostrand (SE); Carl Arne Krister Borrebaeck, Lund (SE); Henrik Johansson, Malmo (SE); Ann-Sofie Albrekt, Teckomatorp (SE)

(73) Assignee: SenzaGen AB, Bunkeflostrand (SE)

(21) Appl. No.: 13/881,576

(22) PCT Filed: Oct. 26, 2011
(86) PCT No.: PCT/GB2011/052082

§ 371 Date: Jul. 9, 2013

(30) Foreign Application Priority Data
Oct. 26, 2010 (GB) 1018014.9

(51) Int. Cl. G01N 33/50

(52) U.S. Cl. CPC G01N 33/5047; G01N 33/5023
USPC 506/9; 435/6.13; 435/6.11; 435/6.12; 506/16

(57) ABSTRACT

The present invention relates to an in vitro method for identifying agents capable of inducing sensitization of human skin and arrays and diagnostic kits for use in such methods. In particular, the methods include measurement of the expression of the biomarkers listed in Table 3A and/or 3B in MUTZ-3 cells exposed to a test agent.

### FIELD OF THE INVENTION

The present invention relates to an in vitro method for identifying agents capable of inducing sensitization of human skin and arrays and analytical kits for use in such methods.

### BACKGROUND

Allergic contact dermatitis is an inflammatory skin disease that affects a significant proportion of the population. It is commonly caused by immunological responses towards chemical haptens leading to substantial economic burden for society. Current tests for sensitizing chemicals rely on animal experimentation. New legislations on the registration and use of chemicals within, e.g. the pharmaceutical and cosmetic industries, have stimulated significant research efforts to develop alternative human cell-based assays for the prediction of sensitization. The aim is to replace animal experiments with in vitro tests displaying a higher predictive power.

Allergic contact dermatitis (ACD) is a common inflammatory skin disease characterized by eczema and recurrent episodes of itching [1]. The disease affects a significant proportion of the population, with prevalence rates of 7.2% to 18.6% in Europe [2, 3], and the incidence is increasing due to repeated exposure to sensitizing chemicals. ACD is a type IV delayed-type hypersensitivity response caused mainly by reactive T helper 1 (Th1) and interferon (IFN)γ producing CD8⁺ T cells at site of contact with small chemical haptens in previously exposed, and immunologically sensitized, individuals [4]. Dendritic cells (DC) in the epidermis initiate the immune reactions by responding to haptens bound to self-molecules and activating T cell-mediated immunity.

The REACH (Registration, Evaluation, and Authorisation of Chemicals) regulation requires that all new and existing chemicals within the European Union, involving approximately 30 000 chemicals, should be tested for hazardous effects [5]. As the identification of potential sensitizers currently requires animal testing, the REACH legislation will have a huge impact on the number of animals needed for testing. Further, the 7th Amendment to the Cosmetics Directive (76/768/EEC) posed a ban on animal tests for the majority of cosmetic ingredients for human use, to be in effect by 2009, with the exceptions of some tests by 2013. Thus, development of reliable in vitro alternatives to experimental animals for the assessment of sensitizing capacity of chemicals is urgent. To date, no non-animal replacements are available for identification of skin sensitizing chemicals, instead the preferred assay is the mouse Local Lymph Node Assay (LLNA) [6], followed by the Guinea pig maximization test (GPMT) [7]. An in vitro alternative to these animal models would preferably exhibit improved reliability, accuracy and importantly correlate to human reactivity.

Dendritic cells (DCs) play key roles in the immune response by bridging the essential connections between innate and adaptive immunity. They can, upon triggering, rapidly produce large amounts of mediators, which influence migration and activation of other cells at the site of inflammation, and selectively respond to various pathogens and environmental factors, by fine-tuning the cellular response through antigen-presentation. Thus, exploring and utilizing the immunological decision-making by DCs during stimulation with sensitizers, could serve as a potent test strategy for prediction of sensitization.

However, multifaceted phenotypes and specialized functions of different DC subpopulations, as well as their wide and scarce distribution, are complicating factors, which impede the employment of primary DCs as a test platform. Hence, there is a real need to establish accurate and reliable in vitro assays that also circumvent the problems associated with variability of and difficulty in obtaining DCs.

### DISCLOSURE OF THE INVENTION

Thus, the development of assays based on the predictability of DC function should preferably rely on alternative cell types or mimics of in vivo DCs. For this purpose, a cell line with DC characteristics would be advantageous, as it constitutes a stable, reproducible and unlimited supply of cells. In terms of DC mimics, differentiated myelomonocytic MUTZ-3 cells are by far the preferred candidate [8]. MUTZ-3 is as an unlimited source of CD34⁺ DC progenitors and it can acquire, upon cytokine stimulation, phenotypes similar to immature DCs or Langerhans-like DCs [9], present antigens through CD1d, MHC class I and II and induce specific T-cell proliferation [8]. MUTZ-3 also displays a mature transcriptional and phenotypic profile upon stimulation with inflammatory mediators [10].

The present inventors have developed a novel test principle for prediction of skin sensitizers. It has surprisingly been found that skin sensitizers can be accurately identified/predicted using DC progenitor cells, such as MUTZ-3 cells, without further differentiation in a process whereby the cells are stimulated with a panel of sensitizing chemicals, non-sensitizing chemicals, and/or other controls (e.g. vehicle controls comprising diluent only, such as DMSO and/or distilled water). This was found to substantially simplify and improve the reproducibility of the procedure.

The transcriptional response to chemical stimulation was assessed with genome-wide profiling. From data analysis, a biomarker signature of 200 transcripts was identified which completely separated the transcriptional response induced by sensitizing chemicals vs. non-sensitizing chemicals and vehicle controls. Further, the potent predictive power of the signature was illustrated, using SVM and ROC curve analysis. The biomarker signature include transcripts involved in relevant biological pathways, such as DC maturation and cytokine responses, which may shed light on the molecular interactions involved in the process of sensitization. In conclusion, a biomarker signature with potent predictive power, which represents a compelling readout for an in vitro assay useful for the identification of human sensitizing chemicals has been identified.

Hence, a first aspect of the present invention provides an in vitro method for identifying agents capable of inducing sensitization of mammalian skin comprising or consisting of the steps of:
a) exposing a population of dendritic cells or a population of dendritic-like cells to a test agent; and
b) measuring in the cells the expression of one or more biomarker(s) selected from the group defined in Table 3,
   wherein the expression in the cells of the one or more biomarkers measured in step (b) is indicative of the sensitizing effect of the test agent.

By “agents capable of inducing sensitization of mammalian skin” we mean any agent capable of inducing and triggering a Type IV delayed-type hypersensitivity reaction in a mammal. Preferably, the Type IV delayed-type hypersensitivity reaction is DC-mediated.

In one embodiment, the “agents capable of inducing sensitization of mammalian skin” is an agent capable of inducing and triggering a Type IV delayed-type hypersensitivity reaction at a site of epidermal contact in a mammal.

The mammal may be any domestic or farm animal. Preferably, the mammal is a rat, mouse, guinea pig, cat, dog, horse or a primate. Most preferably, the mammal is human. As discussed above, in vivo methods of determining sensitisation are known in the art. A preferred method is the Local lymph node assay (for details, see Basketter, D. A., et al., Local lymph node assay—validation, conduct and use in practice. Food Chem Toxicol, 2002. 40(5): p. 593-8). A further suitable, but less preferred, method is the guinea pig maximization test (for details, see Magnusson, B. and A. M. Kligman, The identification of contact allergens by animal assay. The guinea pig maximization test. J Invest Dermatol, 1969. 52(3): p. 268-76).

By “dendritic-like cells” we mean non-dendritic cells that exhibit functional and phenotypic characteristics specific to dendritic cells such as morphological characteristics, expression of costimulatory molecules and MHC class II molecules, and the ability to pinocytose macromolecules and to activate resting T cells.

In one embodiment, the dendritic-like cells are CD34⁺ dendritic cell progenitors. Optionally, the CD34⁺ dendritic cell progenitors can acquire, upon cytokine stimulation, the phenotypes of presenting antigens through CD1d, MHC class I and II, induce specific T-cell proliferation, and/or displaying a mature transcriptional and phenotypic profile upon stimulation with inflammatory mediators (i.e. similar phenotypes to immature dendritic cells or Langerhans-like dendritic cells).

Dendritic cells may be recognized by function, by phenotype and/or by gene expression pattern, particularly by cell surface phenotype. These cells are characterized by their distinctive morphology, high levels of surface MHC-class II expression and ability to present antigen to CD4+ and/or CD8+ T cells, particularly to naïve T cells (Steinman et al. (1991) Ann. Rev. Immunol. 9: 271).

The cell surface of dendritic cells is unusual, with characteristic veil-like projections, and is characterized by expression of the cell surface markers CD11c and MHC class II. Most DCs are negative for markers of other leukocyte lineages, including T cells, B cells, monocytes/macrophages, and granulocytes. Subpopulations of dendritic cells may also express additional markers including 33D1, CCR1, CCR2, CCR4, CCR5, CCR6, CCR7, CD1a-d, CD4, CD5, CD8alpha, CD9, CD11b, CD24, CD40, CD48, CD54, CD58, CD80, CD83, CD86, CD91, CD117, CD123 (IL3Ra), CD134, CD137, CD150, CD153, CD162, CXCR1, CXCR2, CXCR4, DCIR, DC-LAMP, DC-SIGN, DEC205, E-cadherin, Langerin, Mannose receptor, MARCO, TLR2, TLR3TLR4, TLR5, TLR6, TLR9, and several lectins.

The patterns of expression of these cell surface markers may vary along with the maturity of the dendritic cells, their tissue of origin, and/or their species of origin. Immature dendritic cells express low levels of MHC class II, but are capable of endocytosing antigenic proteins and processing them for presentation in a complex with MHC class II molecules. Activated dendritic cells express high levels of MHC class 11, ICAM-1 and CD86, and are capable of stimulating the proliferation of naive allogeneic T cells, e.g. in a mixed leukocyte reaction (MLR).

Functionally, dendritic cells or dendritic-like cells may be identified by any convenient assay for determination of antigen presentation. Such assays may include testing the ability to stimulate antigen-primed and/or naive T cells by presentation of a test antigen, followed by determination of T cell proliferation, release of IL-2, and the like.

By “expression” we mean the level or amount of a gene product such as mRNA or protein.

Methods of detecting and/or measuring the concentration of protein and/or nucleic acid are well known to those skilled in the art, see for example Sambrook and Russell, 2001, Cold Spring Harbor Laboratory Press.

Preferred methods for detection and/or measurement of protein include Western blot, North-Western blot, immunosorbent assays (ELISA), antibody microarray, tissue microarray (TMA), immunoprecipitation, in situ hybridisation and other immunohistochemistry techniques, radioimmunoassay (RIA), immunoradiometric assays (IRMA) and immunoenzymatic assays (IEMA), including sandwich assays using monoclonal and/or polyclonal antibodies. Exemplary sandwich assays are described by David et al., in U.S. Pat. Nos. 4,376,110 and 4,486,530, hereby incorporated by reference. Antibody staining of cells on slides may be used in methods well known in cytology laboratory diagnostic tests, as well known to those skilled in the art.

Typically, ELISA involves the use of enzymes which give a coloured reaction product, usually in solid phase assays. Enzymes such as horseradish peroxidase and phosphatase have been widely employed. A way of amplifying the phosphatase reaction is to use NADP as a substrate to generate NAD which now acts as a coenzyme for a second enzyme system. Pyrophosphatase from Escherichia coli provides a good conjugate because the enzyme is not present in tissues, is stable and gives a good reaction colour. Chemi-luminescent systems based on enzymes such as luciferase can also be used.

Conjugation with the vitamin biotin is frequently used since this can readily be detected by its reaction with enzyme-linked avidin or streptavidin to which it binds with great specificity and affinity.

Preferred methods for detection and/or measurement of nucleic acid (e.g. mRNA) include southern blot, northern blot, polymerase chain reaction (PCR), reverse transcriptase PCR (RT-PCR), quantitative real-time PCR (qRT-PCR), nanoarray, microarray, macroarray, autoradiography and in situ hybridisation.

In one embodiment the method comprises exposing a separate population of the dendritic cells or dendritic-like cells to a negative control agent that does not sensitize human skin, and measuring in the cells the expression of the one or more biomarker(s) measured in step (b). Hence, a sensitizing effect of the test agent is indicated in the event that the expression in the cell population of the one or more biomarker(s) measured in step (b) is/are different from the expression in the negative control sample.

In one embodiment, the negative control agent is a solvent for use with the test or control agents of the invention. Hence, the negative control may be DMSO and/or distilled water.

In another embodiment, the expression of one or more biomarkers measured in step (b) of the dendritic cells or dendritic-like cells prior to test agent exposure is used as a negative control.

A further embodiment comprises exposing a separate population of the dendritic cells or dendritic-like cells to a positive control agent that sensitizes human skin and measuring in the cells the expression of the one or more biomarker(s) measured in step (b). Hence, a sensitizing effect of the test agent is indicated in the event that the expression in the cell population of the one or more biomarker(s) measured in step (b) is/are similar to or the same as the expression in the positive control sample.

Preferably the method comprises, in step (b), measuring the expression of at least one biomarker selected from the group consisting of:
i) taste receptor, type 2, member 5 (TAS2R5),
ii) keratinocyte growth factor-like protein 1/2/hypothetical protein FLJ20444 (KGFLP1/2/FLJ20444),
iii) transmembrane anterior posterior transformation 1 (TAPT1),
iv) sprouty homolog 2 (SPRY2),
v) fatty acid synthase (FASN),
vi) B-cell CLL/lymphoma 7A (BCL7A),
vii) solute carrier family 25, member 32 (SLC25A32),
viii) ferritin, heavy polypeptide pseudogene 1 (FTHP1),
ix) ATPase, H+ transporting, lysosomal 50/57 kDa, V1 subunit H (ATP6V1H),
x) squalene epoxidase (SQLE), and
xi) histone cluster 1, H1e (HIST1H1E).

Hence, in one embodiment the expression of taste receptor, type 2, member 5 (TAS2R5) is measured in step (b). In a further embodiment, in step (b), the expression of keratinocyte growth factor-like protein 1/2/hypothetical protein FLJ20444 (KGFLP1/2/FLJ20444) is measured. The method may comprise measuring the expression of transmembrane anterior posterior transformation 1 (TAPT1) in step (b). In one embodiment, the method comprises measuring the expression of sprouty homolog 2 (SPRY2) in step (b). However, a further embodiment the method, in step (b), comprises measuring the expression of fatty acid synthase (FASN). The method may comprise measuring the expression of B-cell CLL/lymphoma 7A (BCL7A) in step (b). It may also comprise measuring the expression of solute carrier family 25, member 32 (SLC25A32) in step (b). It may additionally comprise, in step (b), measuring the expression of ferritin, heavy polypeptide pseudogene 1 (FTHP1). A still further embodiment comprises measuring the expression of ATPase, H+ transporting, lysosomal 50/57 kDa, V1 subunit H (ATP6V1H) in step (b). In another embodiment step (b) comprises measuring the expression of squalene epoxidase (SQLE). In yet another embodiment the expression of histone cluster 1, H1e (HIST1H1E) is measured in step (b).

The method may comprise or consist of measuring, in step (b), the expression of at least 2 biomarkers from Table 3A, for example, at least 3, 4, 5, 6, 7, 8, 9, 10 or 11 biomarkers from Table 3A. In a preferred embodiment, the method comprises or consists of measuring the expression of fatty acid synthase (FASN) and squalene epoxidase (SQLE) in step (b).

The method may additionally or alternatively comprise or consist of, measuring in step (b) the expression of at least 2 biomarkers from Table 3B, for example, at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, or at least 189 biomarkers from Table 3B.

Thus, the expression of all of the biomarkers in Table 3A and/or all of the biomarkers in Table 3B may be measured in step (b).

In a preferred embodiment, step (b) comprises or consists of measuring the expression of a nucleic acid molecule encoding the one or more biomarker(s). The nucleic acid molecule may be a cDNA molecule or an mRNA molecule. Preferably, the nucleic acid molecule is an mRNA molecule.

In one embodiment the expression of the one or more biomarker(s) in step (b) is performed using a method selected from the group consisting of Southern hybridisation, Northern hybridisation, polymerase chain reaction (PCR), reverse transcriptase PCR (RT-PCR), quantitative real-time PCR (qRT-PCR), nanoarray, microarray, macroarray, autoradiography and in situ hybridisation. Preferably, the expression of the one or more biomarker(s) is measured using a DNA microarray.

The method may comprise measuring the expression of the one or more biomarker(s) in step (b) using one or more binding moieties, each capable of binding selectively to a nucleic acid molecule encoding one of the biomarkers identified in Table 3. In one embodiment the one or more binding moieties each comprise or consist of a nucleic acid molecule. In a further embodiment the one or more binding moieties each comprise or consist of DNA, RNA, PNA, LNA, GNA, TNA or PMO. Preferably, the one or more binding moieties each comprise or consist of DNA. In one embodiment, the one or more binding moieties are 5 to 100 nucleotides in length. However, in an alternative embodiment, they are 15 to 35 nucleotides in length.

Suitable binding agents (also referred to as binding molecules) may be selected or screened from a library based on their ability to bind a given nucleic acid, protein or amino acid motif, as discussed below.

In a preferred embodiment, the binding moiety comprises a detectable moiety.

By a “detectable moiety” we include a moiety which permits its presence and/or relative amount and/or location (for example, the location on an array) to be determined, either directly or indirectly.

Suitable detectable moieties are well known in the art.

For example, the detectable moiety may be a fluorescent and/or luminescent and/or chemiluminescent moiety which, when exposed to specific conditions, may be detected. Such a fluorescent moiety may need to be exposed to radiation (i.e. light) at a specific wavelength and intensity to cause excitation of the fluorescent moiety, thereby enabling it to emit detectable fluorescence at a specific wavelength that may be detected.

Alternatively, the detectable moiety may be an enzyme which is capable of converting a (preferably undetectable) substrate into a detectable product that can be visualised and/or detected. Examples of suitable enzymes are discussed in more detail below in relation to, for example, ELISA assays.

Hence, the detectable moiety may be selected from the group consisting of: a fluorescent moiety; a luminescent moiety; a chemiluminescent moiety; a radioactive moiety (for example, a radioactive atom); or an enzymatic moiety. Preferably, the detectable moiety comprises or consists of a radioactive atom. The radioactive atom may be selected from the group consisting of technetium-99m, iodine-123, iodine-125, iodine-131, indium-111, fluorine-19, carbon-13, nitrogen-15, oxygen-17, phosphorus-32, sulphur-35, deuterium, tritium, rhenium-186, rhenium-188 and yttrium-90.

Clearly, the agent to be detected (such as, for example, the one or more biomarkers in the test sample and/or control sample described herein and/or an antibody molecule for use in detecting a selected protein) must have sufficient of the appropriate atomic isotopes in order for the detectable moiety to be readily detectable.

In an alternative preferred embodiment, the detectable moiety of the binding moiety is a fluorescent moiety.

The radio- or other labels may be incorporated into the biomarkers present in the samples of the methods of the invention and/or the binding moieties of the invention in known ways. For example, if the binding agent is a polypeptide it may be biosynthesised or may be synthesised by chemical amino acid synthesis using suitable amino acid precursors involving, for example, fluorine-19 in place of hydrogen. Labels such as ^{99m}Tc, ¹²³I, ¹⁸⁶Rh, ¹⁸⁸Rh and ¹¹¹In can, for example, be attached via cysteine residues in the binding moiety. Yttrium-90 can be attached via a lysine residue. The IODOGEN method (Fraker et al (1978) Biochem. Biophys. Res. Comm. 80, 49-57) can be used to incorporate ¹²³I. Reference (“Monoclonal Antibodies in Immunoscintigraphy”, J-F Chatal, CRC Press, 1989) describes other methods in detail. Methods for conjugating other detectable moieties (such as enzymatic, fluorescent, luminescent, chemiluminescent or radioactive moieties) to proteins are well known in the art.

It will be appreciated by persons skilled in the art that biomarkers in the sample(s) to be tested may be labelled with a moiety which indirectly assists with determining the presence, amount and/or location of said proteins. Thus, the moiety may constitute one component of a multicomponent detectable moiety. For example, the biomarkers in the sample(s) to be tested may be labelled with biotin, which allows their subsequent detection using streptavidin fused or otherwise joined to a detectable label.

In another embodiment of first aspect of the present invention step (b) comprises determining the expression of the protein of the one or more biomarker(s). The method may comprise measuring the expression of the one or more biomarker(s) in step (b) using one or more binding moieties each capable of binding selectively to one of the biomarkers identified in Table 3. The one or more binding moieties may comprise or consist of an antibody or an antigen-binding fragment thereof such as a monoclonal antibody or fragment thereof.

The term “antibody” includes any synthetic antibodies, recombinant antibodies or antibody hybrids, such as but not limited to, a single-chain antibody molecule produced by phage-display of immunoglobulin light and/or heavy chain variable and/or constant regions, or other immunointeractive molecules capable of binding to an antigen in an immunoassay format that is known to those skilled in the art.

We also include the use of antibody-like binding agents, such as affibodies and aptamers.

A general review of the techniques involved in the synthesis of antibody fragments which retain their specific binding sites is to be found in Winter & Milstein (1991) Nature 349, 293-299.

Additionally, or alternatively, one or more of the first binding molecules may be an aptamer (see Collett et al., 2005, Methods 37:4-15).

Molecular libraries such as antibody libraries (Clackson et al, 1991, Nature 352, 624-628; Marks et al, 1991, J Mol Biol 222(3): 581-97), peptide libraries (Smith, 1985, Science 228(4705): 1315-7), expressed cDNA libraries (Santi et al (2000) J Mol Biol 296(2): 497-508), libraries on other scaffolds than the antibody framework such as affibodies (Gunneriusson et al, 1999, Appl Environ Microbiol 65(9): 4134-40) or libraries based on aptamers (Kenan et al, 1999, Methods Mol Biol 118, 217-31) may be used as a source from which binding molecules that are specific for a given motif are selected for use in the methods of the invention.

The molecular libraries may be expressed in vivo in prokaryotic cells (Clackson et al, 1991, op. cit.; Marks et al, 1991, op. cit.) or eukaryotic cells (Kieke et al., 1999, Proc Natl Acad Sci USA, 96(10):5651-6) or may be expressed in vitro without involvement of cells (Hanes & Pluckthun, 1997, Proc Natl Acad Sci USA 94(10):4937-42; He & Taussig, 1997, Nucleic Acids Res 25(24):5132-4; Nemoto et al, 1997, FEBS Lett, 414(2):405-8).

In cases when protein based libraries are used, the genes encoding the libraries of potential binding molecules are often packaged in viruses and the potential binding molecule displayed at the surface of the virus (Clackson et al, 1991, supra; Marks et al, 1991, supra; Smith, 1985, supra).

Perhaps the most commonly used display system is filamentous bacteriophage displaying antibody fragments at their surfaces, the antibody fragments being expressed as a fusion to the minor coat protein of the bacteriophage (Clackson et al, 1991, supra; Marks et al, 1991, supra). However, other suitable systems for display include using other viruses (EP 39578), bacteria (Gunneriusson et al, 1999, supra; Daugherty et al, 1998, Protein Eng 11(9):825-32; Daugherty et al, 1999, Protein Eng 12(7):613-21), and yeast (Shusta et al, 1999, J Mol Biol 292(5):949-56).

In addition, display systems have been developed utilising linkage of the polypeptide product to its encoding mRNA in so-called ribosome display systems (Hanes & Pluckthun, 1997, supra; He & Taussig, 1997, supra; Nemoto et al, 1997, supra), or alternatively linkage of the polypeptide product to the encoding DNA (see U.S. Pat. No. 5,856,090 and WO 98/37186).

The variable heavy (V_{H}) and variable light (V_{L}) domains of the antibody are involved in antigen recognition, a fact first recognised by early protease digestion experiments. Further confirmation was found by “humanisation” of rodent antibodies. Variable domains of rodent origin may be fused to constant domains of human origin such that the resultant antibody retains the antigenic specificity of the rodent parented antibody (Morrison et al (1984) Proc. Natl. Acad. Sci. USA 81, 6851-6855).

That antigenic specificity is conferred by variable domains and is independent of the constant domains is known from experiments involving the bacterial expression of antibody fragments, all containing one or more variable domains. These molecules include Fab-like molecules (Better et al (1988) Science 240, 1041); Fv molecules (Skerra et al (1988) Science 240, 1038); single-chain Fv (ScFv) molecules where the V_{H} and V_{L} partner domains are linked via a flexible oligopeptide (Bird et al (1988) Science 242, 423; Huston et al (1988) Proc. Natl. Acad. Sci. USA 85, 5879) and single domain antibodies (dAbs) comprising isolated V domains (Ward et al (1989) Nature 341, 544). A general review of the techniques involved in the synthesis of antibody fragments which retain their specific binding sites is to be found in Winter & Milstein (1991) Nature 349, 293-299.

The antibody or antigen-binding fragment may be selected from the group consisting of intact antibodies, Fv fragments (e.g. single chain Fv and disulphide-bonded Fv), Fab-like fragments (e.g. Fab fragments, Fab′ fragments and F(ab)₂ fragments), single variable domains (e.g. V_{H} and V_{L} domains) and domain antibodies (dAbs, including single and dual formats [i.e. dAb-linker-dAb]). Preferably, the antibody or antigen-binding fragment is a single chain Fv (scFv).

The one or more binding moieties may alternatively comprise or consist of an antibody-like binding agent, for example an affibody or aptamer.

By “scFv molecules” we mean molecules wherein the V_{H} and V_{L} partner domains are linked via a flexible oligopeptide.

The advantages of using antibody fragments, rather than whole antibodies, are several-fold. The smaller size of the fragments may lead to improved pharmacological properties, such as better penetration of solid tissue. Effector functions of whole antibodies, such as complement binding, are removed. Fab, Fv, ScFv and dAb antibody fragments can all be expressed in and secreted from E. coli, thus allowing the facile production of large amounts of the said fragments.

Whole antibodies, and F(ab′)₂ fragments are “bivalent”. By “bivalent” we mean that the said antibodies and F(ab′)₂ fragments have two antigen combining sites. In contrast, Fab, Fv, ScFv and dAb fragments are monovalent, having only one antigen combining sites.

The antibodies may be monoclonal or polyclonal. Suitable monoclonal antibodies may be prepared by known techniques, for example those disclosed in “Monoclonal Antibodies: A manual of techniques”, H Zola (CRC Press, 1988) and in “Monoclonal Hybridoma Antibodies: Techniques and applications”, J G R Hurrell (CRC Press, 1982), both of which are incorporated herein by reference.

When potential binding molecules are selected from libraries, one or more selector peptides having defined motifs are usually employed. Amino acid residues that provide structure, decreasing flexibility in the peptide or charged, polar or hydrophobic side chains allowing interaction with the binding molecule may be used in the design of motifs for selector peptides. For example:
(i) Proline may stabilise a peptide structure as its side chain is bound both to the alpha carbon as well as the nitrogen;
(ii) Phenylalanine, tyrosine and tryptophan have aromatic side chains and are highly hydrophobic, whereas leucine and isoleucine have aliphatic side chains and are also hydrophobic;
(iii) Lysine, arginine and histidine have basic side chains and will be positively charged at neutral pH, whereas aspartate and glutamate have acidic side chains and will be negatively charged at neutral pH;
(iv) Asparagine and glutamine are neutral at neutral pH but contain a amide group which may participate in hydrogen bonds;
(v) Serine, threonine and tyrosine side chains contain hydroxyl groups, which may participate in hydrogen bonds.

Typically, selection of binding molecules may involve the use of array technologies and systems to analyse binding to spots corresponding to types of binding molecules.

The one or more protein-binding moieties may comprise a detectable moiety. The detectable moiety may be selected from the group consisting of a fluorescent moiety, a luminescent moiety, a chemiluminescent moiety, a radioactive moiety and an enzymatic moiety.

In a further embodiment of the methods of the invention, step (b) may be performed using an assay comprising a second binding agent capable of binding to the one or more proteins, the second binding agent also comprising a detectable moiety. Suitable second binding agents are described in detail above in relation to the first binding agents.

Thus, the proteins of interest in the sample to be tested may first be isolated and/or immobilised using the first binding agent, after which the presence and/or relative amount of said biomarkers may be determined using a second binding agent.

In one embodiment, the second binding agent is an antibody or antigen-binding fragment thereof; typically a recombinant antibody or fragment thereof. Conveniently, the antibody or fragment thereof is selected from the group consisting of: scFv; Fab; a binding domain of an immunoglobulin molecule. Suitable antibodies and fragments, and methods for making the same, are described in detail above.

Alternatively, the second binding agent may be an antibody-like binding agent, such as an affibody or aptamer.

Alternatively, where the detectable moiety on the protein in the sample to be tested comprises or consists of a member of a specific binding pair (e.g. biotin), the second binding agent may comprise or consist of the complimentary member of the specific binding pair (e.g. streptavidin).

Where a detection assay is used, it is preferred that the detectable moiety is selected from the group consisting of: a fluorescent moiety; a luminescent moiety; a chemiluminescent moiety; a radioactive moiety; an enzymatic moiety. Examples of suitable detectable moieties for use in the methods of the invention are described above.

Preferred assays for detecting serum or plasma proteins include enzyme linked immunosorbent assays (ELISA), radioimmunoassay (RIA), immunoradiometric assays (IRMA) and immunoenzymatic assays (IEMA), including sandwich assays using monoclonal and/or polyclonal antibodies. Exemplary sandwich assays are described by David et al in U.S. Pat. Nos. 4,376,110 and 4,486,530, hereby incorporated by reference. Antibody staining of cells on slides may be used in methods well known in cytology laboratory diagnostic tests, as well known to those skilled in the art.

Thus, in one embodiment the assay is an ELISA (Enzyme Linked Immunosorbent Assay) which typically involves the use of enzymes which give a coloured reaction product, usually in solid phase assays. Enzymes such as horseradish peroxidase and phosphatase have been widely employed. A way of amplifying the phosphatase reaction is to use NADP as a substrate to generate NAD which now acts as a coenzyme for a second enzyme system. Pyrophosphatase from Escherichia coli provides a good conjugate because the enzyme is not present in tissues, is stable and gives a good reaction colour. Chemiluminescent systems based on enzymes such as luciferase can also be used.

Conjugation with the vitamin biotin is frequently used since this can readily be detected by its reaction with enzyme-linked avidin or streptavidin to which it binds with great specificity and affinity.

In an alternative embodiment, the assay used for protein detection is conveniently a fluorometric assay. Thus, the detectable moiety of the second binding agent may be a fluorescent moiety, such as an Alexa fluorophore (for example Alexa-647).

Preferably, step (b) is performed using an array. The array may be a bead-based array or a surface-based array. The array may be selected from the group consisting of: macroarray; microarray; nanoarray.

In on embodiment, the method is for identifying agents capable of inducing a hypersensitivity response in human skin. Preferably, the hypersensitivity response is a cell-mediated hypersensitivity response, for example, a type IV hypersensitivity response. Preferably, the method is for identifying agents capable of inducing allergic contact dermatitis (ACD) (i.e. the hypersensitivity response is ACD).

In one embodiment, the population of dendritic cells or population of dendritic-like cells is a population of dendritic cells. Preferably, the dendritic cells are primary dendritic cells. Preferably, the dendritic cells are myeloid dendritic cells.

The population of dendritic cells or dendritic-like cells is preferably mammalian in origin. Preferably, the mammal is a rat, mouse, guinea pig, cat, dog, horse or a primate. Most preferably, the mammal is human.

In an embodiment the population of dendritic cells or population of dendritic-like cells is a population of dendritic-like cells, preferably myeloid dendritic-like cells.

In one embodiment, the dendritic-like cells express at least one of the markers selected from the group consisting of CD54, CD86, CD80, HLA-DR, CD14, CD34 and CD1a, for example, 2, 3, 4, 5, 6 or 7 of the markers. In a further embodiment, the dendritic-like cells express the markers CD54, CD86, CD80, HLA-DR, CD14, CD34 and CD1a.

In a further embodiment, the dendritic-like cells may be derived from myeloid dendritic cells. Preferably the dendritic-like cells are myeloid leukaemia-derived cells. Preferably, the myeloid leukaemia-derived cells are selected from the group consisting of KG-1, THP-1, U-937, HL-60, Monomac-6, AML-193 and MUTZ-3. Most preferably, dendritic-like cells are MUTZ-3 cells. MUTZ-3 cells are human acute myelomonocytic leukemia cells that were deposited with Deutsche Sammlung für Mikroorganismen and Zeilkulturen GmbH (DSMZ), (Inhoffenstraβe 7B, Braunschweig, Germany) on 15 May 1995 (www.dsmz.de; deposit no. ACC 295).

In one embodiment, the dendritic-like cells, after stimulation with cytokine, present antigens through CD1d, MHC class I and II and/or induce specific T-cell proliferation.

In one embodiment, the negative control agent(s) is/are selected from the group consisting of 1-Butanol, 4-Aminobenzoic acid, Benzaldehyde, Chlorobenzene, Diethyl phthalate, Dimethyl formamide, Ethyl vanillin, Glycerol, Isopropanol, Lactic acid, Methyl salicylate, Octanoic acid, Propylene glycol, Phenol, p-ydroxybenzoic add, Potassium permanganate, Salicylic acid, Sodium dodecyl sulphate, Tween 80 and Zinc sulphate.

The method may comprise the use of at least 2 negative control agents (i.e. non-sensitizing agents), for example, at least 3, 4, 5, 6, 7, 8, 9, 10, 11, 12, 13, 14, 15, 16, 17, 16, 19 or at least 20 negative control agents.

In another embodiment, the positive control agent(s) is/are selected from the group consisting of 2,4-Dinitrochlorobenzene, Oxazolone, Potassium dichromate, Kathon CH (MC/MCI), Formaldehyde, 2-Aminophenol, 2-nitro-1,4-Phenylendiamine, p-Phenylendiamine, Hexylcinnamic aldehyde, 2-Hydroxyethyl acrylate, 2-Mercaptobenzothiazole, Glyoxal, Cinnamaldehyde, Isoeugenol, Ethylendiamine, Resorcinol, Cinnamic alcohol, Eugenol, Penicillin G or Geraniol.

The method may comprise the use of at least 2 positive control (i.e. sensitizing agents) are provided, for example, at least 3, 4, 5, 6, 7, 8, 9, 10, 11, 12, 13, 14, 15, 16, 17, 18, 19 or at least 20 positive control agents.

In one embodiment, the method is indicative of whether the test agent is or is not a sensitizing agent. In alternative or additional embodiment, the method is indicative of the sensitizing potency of the sample to be tested.

In one embodiment the method is indicative of the local lymph node assay (LLNA) classification of the sensitizing potency of the sample to be tested. For a detailed description of LLNA see Basketter, D. A., et al., Local lymph node assay—validation, conduct and use in practice. Food Chem Toxicol, 2002. 40(5): p. 593-8 which is incorporated herein by reference.

In an alternative embodiment, the method is indicative of the guinea pig maximization test classification of the sensitizing potency of the sample to be tested. For a detailed description of the guinea pig maximization test see Magnusson, B. and A. M. Kligman, The identification of contact allergens by animal assay. The guinea pig maximization test. J Invest Dermatol, 1969. 52(3): p. 268-76, which is incorporated herein by reference.

Thus, in one embodiment, the method is indicative that the test agent is either, a non-sensitizer, a weak sensitizer, a moderate sensitizer, a strong sensitizer or an extreme sensitizer. The decision value and distance in PCA correlates with sensitizer potency.

Generally, skin sensitizing agents are determined with an ROC AUC of at least 0.55, for example with an ROC AUC of at least, 0.60, 0.65, 0.70, 0.75, 0.80, 0.85, 0.90, 0.95, 0.96, 0.97, 0.98, 0.99 or with an ROC AUC of 1.00. Preferably, skin sensitizing agents are determined with an ROC AUC of at least 0.85, and most preferably with an ROC AUC of 1.

Typically, agents capable of inducing sensitization are identified using a support vector machine (SVM), such as those available from http://cran.r-project.org/web/packages/e1071/index.html (e.g. e1071 1.5-24). However, any other suitable means may also be used.

Support vector machines (SVMs) are a set of related supervised learning methods used for classification and regression. Given a set of training examples, each marked as belonging to one of two categories, an SVM training algorithm builds a model that predicts whether a new example falls into one category or the other. Intuitively, an SVM model is a representation of the examples as points in space, mapped so that the examples of the separate categories are divided by a clear gap that is as wide as possible. New examples are then mapped into that same space and predicted to belong to a category based on which side of the gap they fall on.

More formally, a support vector machine constructs a hyperplane or set of hyperplanes in a high or infinite dimensional space, which can be used for classification, regression or other tasks. Intuitively, a good separation is achieved by the hyperplane that has the largest distance to the nearest training datapoints of any class (so-called functional margin), since in general the larger the margin the lower the generalization error of the classifier. For more information on SVMs, see for example, Burges, 1998, Data Mining and Knowledge Discovery, 2:121-167.

In one embodiment of the invention, the SVM is ‘trained’ prior to performing the methods of the invention using biomarker profiles of known agents (namely, known sensitizing or non-sensitizing agents). By running such training samples, the SVM is able to learn what biomarker profiles are associated with agents capable of inducing sensitization. Once the training process is complete, the SVM is then able whether or not the biomarker sample tested is from a sensitizing agent or a non-sensitizing agent.

This allows test agents to be classified as sensitizing or non-sensitizing. Moreover, by training the SVM with sensitizing agents of known potency (i.e. non-sensitizing, weak, moderate, strong or extreme sensitizing agents), the potency of test agents can also be identified comparatively.

However, this training procedure can be by-passed by pre-programming the SVM with the necessary training parameters. For example, agents capable of inducing sensitization be identified according to the known SVM parameters using the SVM algorithm detailed in Table 5, based on the measurement of all the biomarkers listed in Table 3(A) and 1(B).

It will be appreciated by skilled persons that suitable SVM parameters can be determined for any combination of the biomarkers listed Table 3 by training an SVM machine with the appropriate selection of data (i.e. biomarker measurements from cells exposed to known sensitizing and/or non-sensitizing agents).

Preferably, the method of the invention has an accuracy of at least 73%, for example 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% accuracy.

Preferably, the method of the invention has a sensitivity of at least 73%, for example 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sensitivity.

Preferably, the method of the invention has a specificity of at least 68%, for example 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% specificity.

By “accuracy” we mean the proportion of correct outcomes of a method, by “sensitivity” we mean the proportion of all positive chemicals that are correctly classified as positives, and by “specificity” we mean the proportion of all negative chemicals that are correctly classified as negatives.

A second aspect of the invention provides an array for use in the method of the first aspect of the invention (or any embodiment or combination of embodiments thereof), the array comprising one or more binding moieties as defined above. In one embodiment, the binding moieties are (collectively) capable of binding to all of the biomarkers defined in Table 3A. In a further embodiment, the binding moieties are (collectively) capable of binding to all of the biomarkers defined in Table 3B. Preferably, the binding moieties are (collectively) capable of binding to all of the biomarkers defined in Table 3A and Table 3B.

The binding moieties may be immobilised.

Arrays per se are well known in the art. Typically they are formed of a linear or two-dimensional structure having spaced apart (i.e. discrete) regions (“spots”), each having a finite area, formed on the surface of a solid support. An array can also be a bead structure where each bead can be identified by a molecular code or colour code or identified in a continuous flow. Analysis can also be performed sequentially where the sample is passed over a series of spots each adsorbing the class of molecules from the solution. The solid support is typically glass or a polymer, the most commonly used polymers being cellulose, polyacrylamide, nylon, polystyrene, polyvinyl chloride or polypropylene. The solid supports may be in the form of tubes, beads, discs, silicon chips, microplates, polyvinylidene difluoride (PVDF) membrane, nitrocellulose membrane, nylon membrane, other porous membrane, non-porous membrane (e.g. plastic, polymer, perspex, silicon, amongst others), a plurality of polymeric pins, or a plurality of microtitre wells, or any other surface suitable for immobilising proteins, polynucleotides and other suitable molecules and/or conducting an immunoassay. The binding processes are well known in the art and generally consist of cross-linking covalently binding or physically adsorbing a protein molecule, polynucleotide or the like to the solid support. Alternatively, affinity coupling of the probes via affinity-tags or similar constructs may be employed. By using well-known techniques, such as contact or non-contact printing, masking or photolithography, the location of each spot can be defined. For reviews see Jenkins, R. E., Pennington, S. R. (2001, Proteomics, 2, 13-29) and Lal et al (2002, Drug Discov Today 15; 7(18 Suppl):S143-9).

Typically the array is a microarray. By “microarray” we include the meaning of an array of regions having a density of discrete regions of at least about 100/cm², and preferably at least about 1000/cm². The regions in a microarray have typical dimensions, e.g. diameter, in the range of between about 10-250 μm, and are separated from other regions in the array by about the same distance. The array may alternatively be a macroarray or a nanoarray.

Once suitable binding molecules (discussed above) have been identified and isolated, the skilled person can manufacture an array using methods well known in the art of molecular biology; see Examples below.

A third aspect of the present invention provides the use of one or more (preferably two or more) biomarkers selected from the group defined in Table 3A and/or Table 3B in combination for identifying hypersensitivity response sensitising agents. Preferably, all of the biomarkers defined in Table 3A and Table 3B are used collectively for identifying hypersensitivity response sensitising agents. Preferably, the use is consistent with the method described in the first aspect of the invention, and the embodiments described therein.

A fourth aspect of the invention provides an analytical kit for use in a method according the first aspect of the invention, comprising or consisting of:
A) an array according to the second aspect of the invention; and
B) instructions for performing the method according to the first aspect of the invention (optional).

The analytical kit may comprise one or more control agents. Preferably, the analytical kit comprises or consists of the above features, together with one or more negative control agents and/or one or more positive control agents.

Preferred, non-limiting examples which embody certain aspects of the invention will now be described, with reference to the following figures:

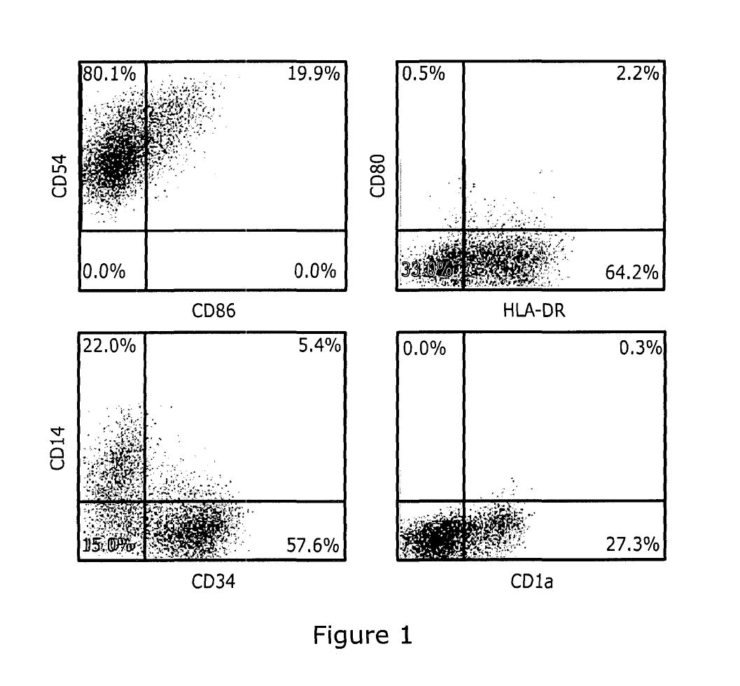

FIG. 1. Phenotype of MUTZ-3 cells prior to stimulation with sensitizing and non-sensitizing chemicals

Cell surface expression levels of CD14, CD1a, CD34, CD54, CD80, CD86 and HLA-DR were assessed with flow cytometry. Gates were set to exclude debris and dead cells, and quadrants were established by comparing with relevant isotype controls. Results are shown from one representative experiment out of six.

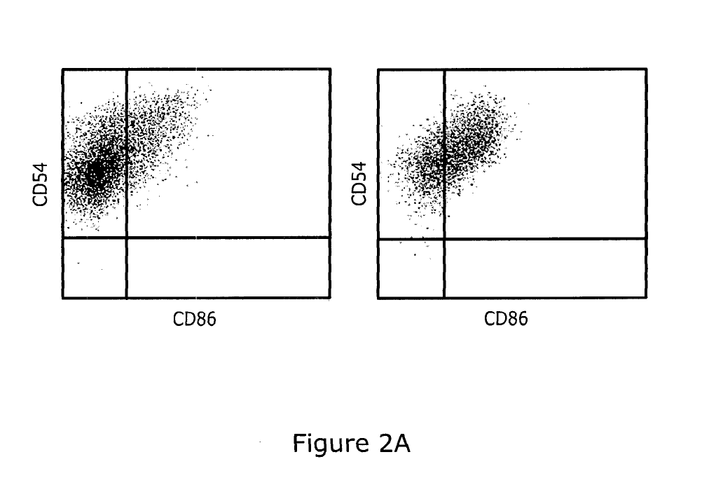

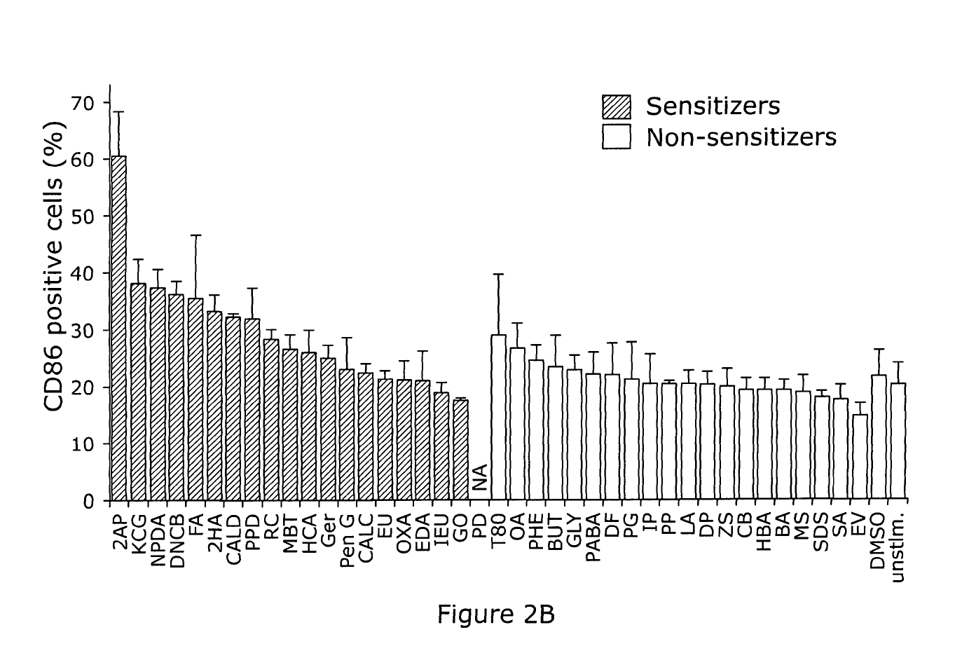

FIG. 2. Changes in CD86 expression following stimulation with sensitizing and non-sensitizing chemicals

Cell surface expression levels of CD86 were monitored after stimulation with chemicals for 24 h. A). Chemical-induced upregulation of CD86, in terms of changes in frequency of positive cells, were determined by flow cytometry, as exemplified by the comparison of 2-aminophenol-stimulated cells (right dotplot) and unstimulated controls (left dotplot). Results are shown from one representative experiment out of three. Gates were set to exclude debris and dead cells, and quadrants were established by comparing with relevant isotype controls. B) Compilation of frequencies of CD86-positive cells after 24 h of stimulation. Statistical analysis was performed using paired Student's t test. *p<0.05, #p<0.01. NA, not analysed.

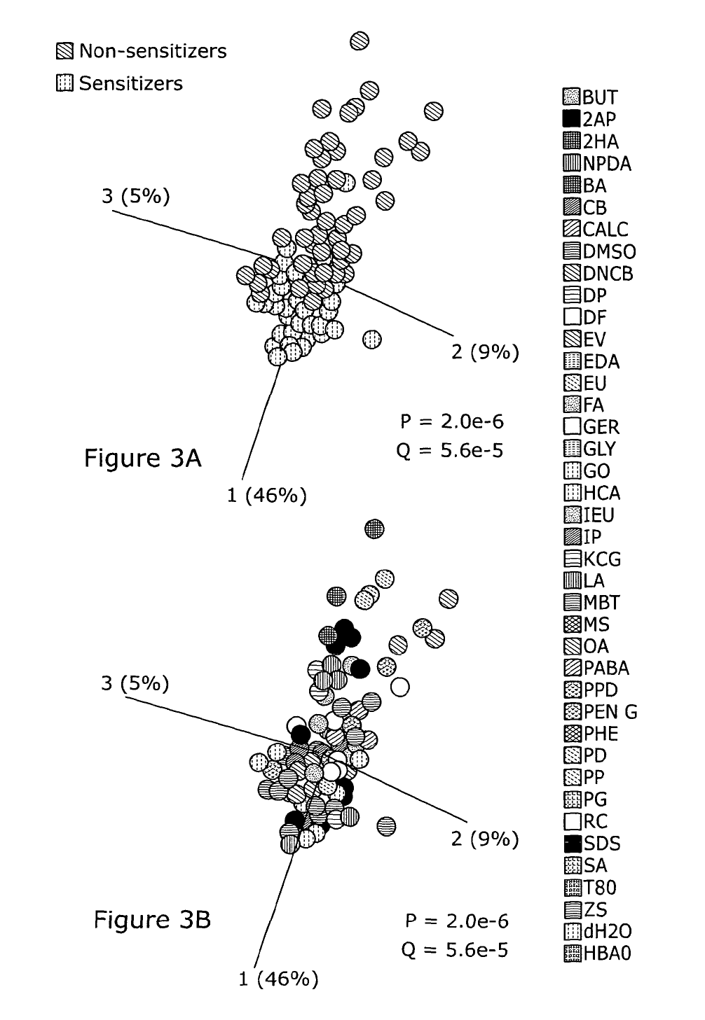

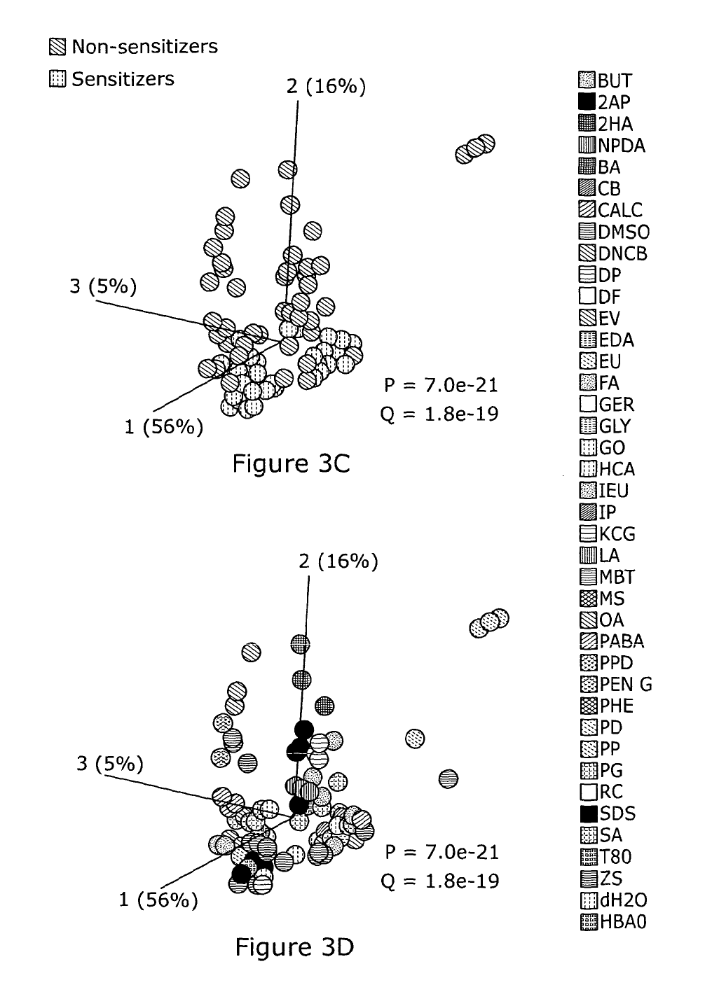

FIG. 3. Principal component analysis of transcripts differentially expressed after chemical stimulation

mRNA levels in MUTZ-3 cells stimulated for 24 h with 20 sensitizing and 20 non-sensitizing chemicals were assessed with transcritomics using Affymetrix Human Gene 1.0 ST arrays. Structures and similarities in the gene expression dataset were investigated using principal component analysis (PCA) in the software Qlucore. A) PCA of genes differentially expressed in cells stimulated with sensitizing (red) as compared to non-sensitizing (green) chemicals (1010 genes identified with one-way ANOVA). B) PCA of genes differentially expressed in cells stimulated with sensitizing as compared to non-sensitizing chemicals (1010 genes), but now samples are coloured by the compound used for stimulation. C) PCA of genes differentially expressed when comparing the different stimulations with 2-way ANOVA (1137 genes). Samples are coloured according to sensitizing (red) and non-sensitizing (green) chemicals. D) PCA of genes differentially expressed when comparing the different stimulations with 2-way ANOVA (1137 genes), but now samples are coloured by the compound used for stimulation. P, p-value from ANOVA. Q, p-value corrected for multiple hypothesis testing.

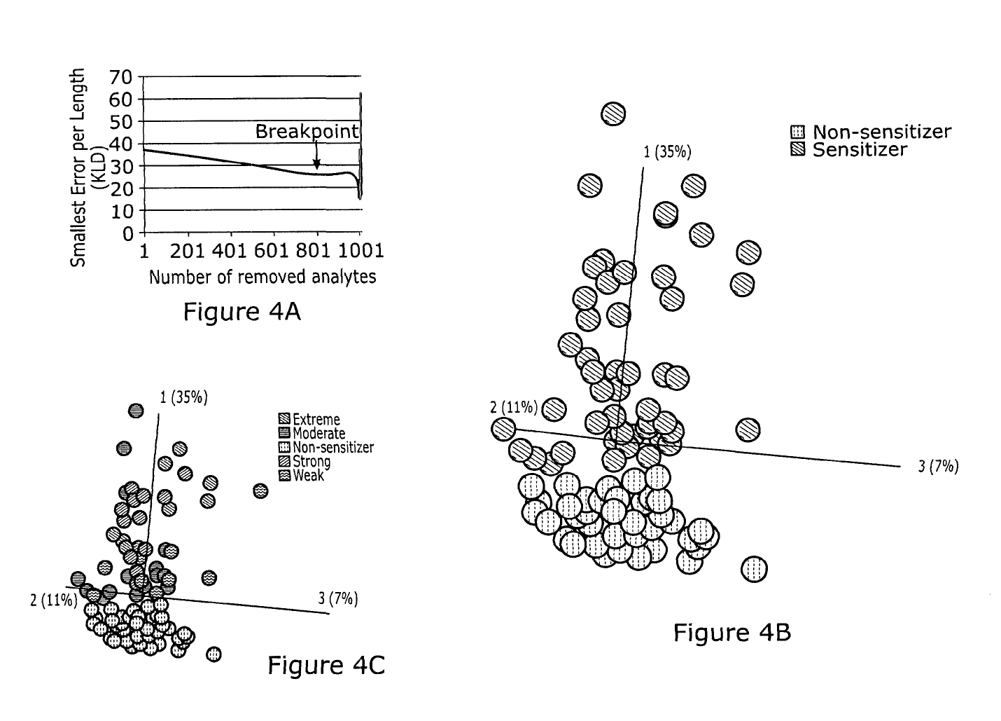

FIG. 4. Identification and PCA analysis of “prediction signature”

A) The number of differentially expressed significant genes in cells stimulated with sensitizing as compared to non-sensitizing chemicals (1010 genes) was reduced using Backward Elimination. The lowest KLD is observed after elimination of 810 analytes, referred to as the Breakpoint. The remaining 200 genes is considered to be the top predictors in the data set, and is termed “Prediction Signature”. B) Complete separation between sensitizers (red) and non-sensitizers (green) is observed with PCA of the “Prediction Signature”. C) Same PCA as in B, now with samples coloured according to their potency in LLNA.

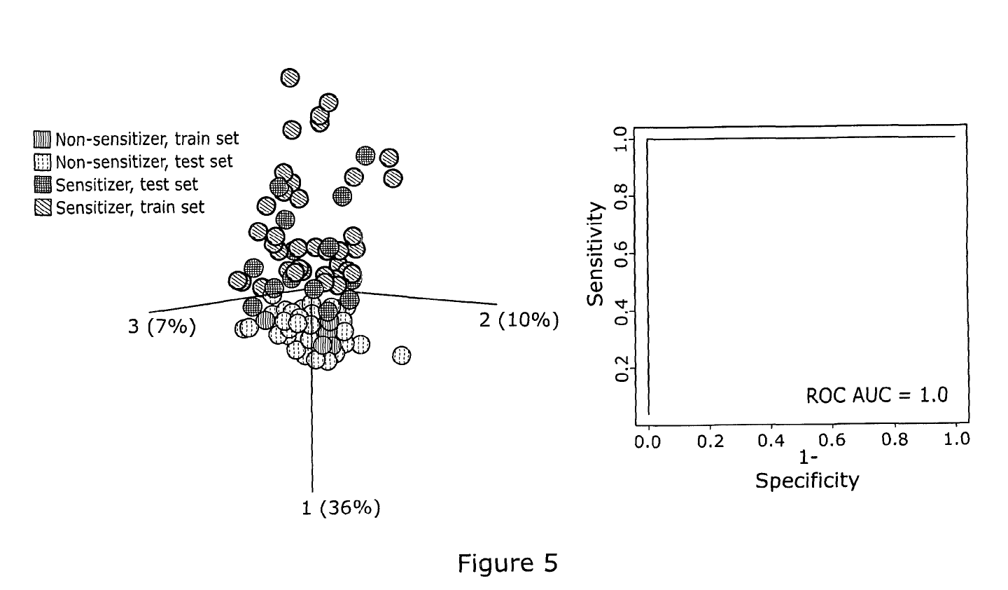

FIG. 5. Validation of selection procedure of “prediction signature”

The method by which the “Prediction Signature” was constructed was validated by repeating the process on 70% of the data set, selected at random. The remaining 30% of data was used as a test set for signature validation. A) PCA demonstrates that the “Test Gene Signature” can separate sensitizers from non-sensitizers. Only the samples of the 70% training set, displayed in bright colours, were used to build the space of the first three principal components. The test set samples, displayed in dark colours, were plotted into this space based on expression levels of the analytes in the “Test Gene Signature”. B) An SVM was trained on the 70% training set, and validated with the 30% test set. The area under the ROC curve of 1.0 proves that the group belonging of all samples in the test set was correctly predicted, demonstrating the strength of the “Test Gene Signature”, and by association also the strength of our “Prediction Signature”.

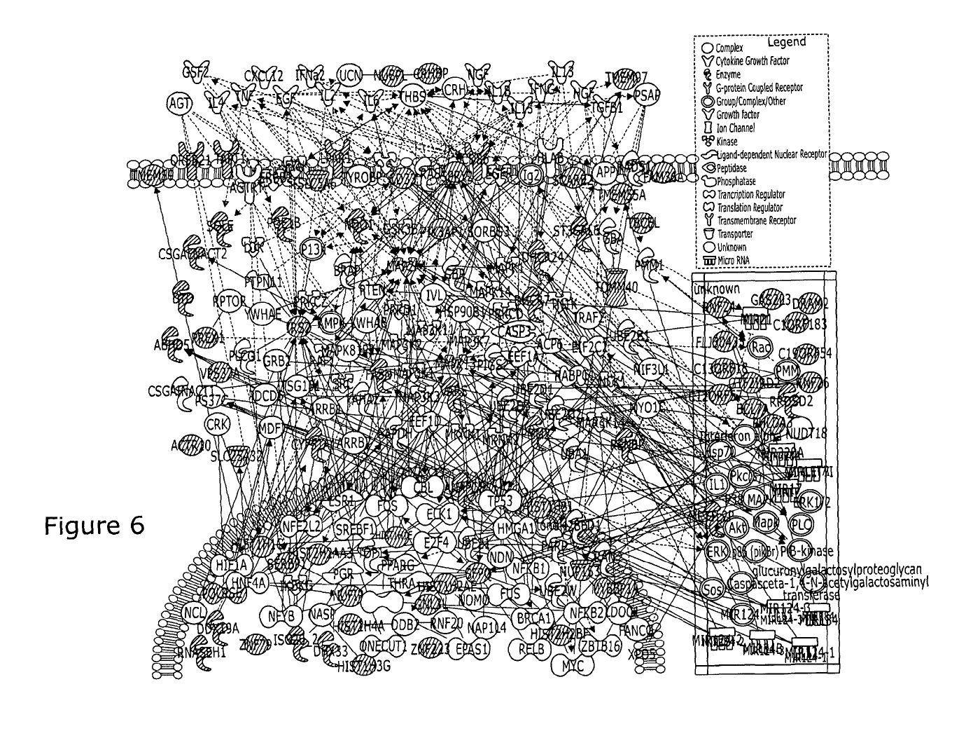

FIG. 6. The interactome of the “prediction signature”

Interactome of the 200 molecules (orange) and molecules connecting theses according to evidence from IPA. Direct interactions are shown as solid lines and indirect as dotted lines.

### EXAMPLES

Allergic contact dermatitis is an inflammatory skin disease that affects a significant proportion of the population. This is commonly caused by immunological responses towards chemical haptens leading to substantial economic burden for society. Current test of sensitizing chemicals rely on animal experimentation. New legislations on the registration and use of chemicals within e.g. pharmaceutical and cosmetic industries have stimulated significant research efforts to develop alternative human cell-based assays for the prediction of sensitization. The aim is to replace animal experiments with in vitro tests displaying a higher predictive power.

We have developed a novel cell-based assay for the prediction of sensitizing chemicals. By analyzing the transcriptome of the human cell line MUTZ-3 after 24 h stimulation with 20 different sensitizing chemicals, 20 non-sensitizing chemicals and vehicle controls, we have identified a biomarker signature of 200 genes with potent discriminatory ability. Using a Support Vector Machine for supervised classification, the prediction performance of the assay revealed an accuracy of 100%, sensitivity of 100% and specificity of 100%. In addition, categorizing the chemicals according to the LLNA assay, this gene signature could also predict potency. The identified markers are involved in biological pathways with immunological relevant functions, which can shed light on the process of human sensitization.

A gene signature predicting sensitization using a human cell line in vitro has been developed. This easy and robust cell-based assay can completely replace or drastically reduce current test systems, using experimental animals. Being based on human biology, the assay is considered to be more relevant and more accurate for predicting sensitization in humans than the traditional animal-based tests.

### Results

### The Cellular Rational of the In Vitro Cell Culture System

Acting as the link between innate and adaptive immunity, DCs are essential immunoregulatory cells of the immune system. Their unique property to recognize antigen for the purpose of initiating T cell responses, and their potent regulatory function in skewing immune responses, makes them targets for assay development. However, primary DCs constitute a heterogeneous and minor population of cells not suited for screening. The obvious advantages of using a cell line with characteristics compared to primary DCs for the basis of a predictive test are stability, reproducibility and unlimited supply of cells. So far, no leukemia with obvious DC-like properties has been reported, probably due to the fact that the characteristics of this cell type are determined by a complex terminal differentiation process that can occur only post-cell division [11], and thus, the generation of DC-like cell lines relies on available myeloid leukemia cell lines. MUTZ-3 is a human acute myelomonocytic leukemia cell line with a potent ability to differentiate into DCs, present antigens and induce specific T-cell proliferation. Among the available myeloid human cell lines, MUTZ-3 is by far the preferred candidate. Similar to immature primary DCs, MUTZ-3 progenitor express CD1a, HLA-DR and CD54, as well as low levels of CD80 and CD86 (FIG. 1). The MUTZ-3 population also contains three subpopulations of CD14⁺, CD34⁺ and double negative cells, previously reported to be transitional differentiation steps from a proliferative CD34⁺ progenitor into a non-proliferative CD14⁺ DC precursor [9]. Consequently, we utilized constitutively differentiating progenitor MUTZ-3 cells as the basis for the test system.

### CD86 Surface Expression in Response to Sensitizer Stimulation:

CD86 is the most extensively studied biomarker for sensitization to date, in cell-systems such as monocyte derived dendritic cells (MoDCs) or dendritic cell-like human cell lines and their progenitors, such as THP-1, U-937 and KG-1. Thus, as a reference, cell surface expression of CD86 was measured with flow cytometry after 24 h stimulation with 20 sensitizers and 20 non-sensitizers, as well as with vehicle controls (Table 1). CD86 was significantly up-regulated on cells stimulated with 2-Aminophenol, Kathon CG, 2-nitro-1,4-Phenylendiamine, 2,4-Dinitrochlorobenzene, 2-Hydroxyethyl acrylate, Cinnamic aldehyde, p-Phenylendiamine, Resorcinol, and 2-Mercaptobenzothiazole. Hence, an assay based on measurement of a single biomarker, such as CD86, would give a sensitivity of 47% and a specificity of 100%. Consequently, CD86 cannot classify skin sensitizers, using a cell based system such as MUTZ-3.

### Analysis of the Transcriptional Profiles in Chemically Stimulated MUTZ-3 Cells:

The genomic expression arrays were used to test 20 sensitizers and 20 non-sensitizers, in triplicates, and vehicle controls such as DMSO and distilled water, the latter in twelve replicates. In total, a data set was generated based on 144 samples. RMA normalization and quality controls of the samples revealed that the Oxazolone and Cinnamic aldehyde samples were significant outliers and had to be removed, or they would have dominated the data set prohibiting biomarker identification (data not shown). In addition, one of the replicates of potassium permanganate had to be removed due to a faulty array. This left a data set consisting of 137 samples, each with data from measurements of 33,297 transcripts. In order to mine the data set for information specific for sensitizers vs. non-sensitizers, the software Qlucore Omics Explorer 2.1 was used, which enable real time principal component analysis (PCA) analysis, while sorting the input genes after desired criteria, e.g. sensitizers and non-sensitizers, based on ANOVA p-value selection. FIGS. 3A and 3B show PCAs based on 1010 transcripts with a p-value of ≦5 2.0×10⁻⁶ from ANOVA analysis, comparing sensitizing vs. non-sensitizing chemicals. As can be seen in FIG. 3A, a clear distinction can be made between the two groups, with non-sensitizers forming a condensed cloud in the lower part of the figure (green), while sensitizers stretch upwards in various directions (red). However, a complete separation is not achieved between the two groups at this level of significance. From FIG. 3B, now coloured according to stimulation, it is evident that one or more replicate of Glyoxal, Eugenol, Hexylcinnamic aldehyde, Isoeugenol, Resorcinol, Penicillin G and Ethylendiamine group together with the control group. In addition, one replicate or more of the non-sensitizers Tween 80, Octanoic acid and Phenol tend to group with the sensitizers. FIGS. 3C and 3D show PCA plots based on 1137 genes that all have a p-value of ≦7.0×10⁻²¹, when comparing the different stimulations. Identifying this large number of genes at this level of significance provides strong indications of the power of the data set. In FIG. 3D, it is clear that the replicates group together, indicating high quality data. The triplicate samples of Potassium dichromate have a discrete profile, which demonstrate a substantial impact of the cells compared to non-sensitizers. Furthermore, 2-Hydroxyethyl acrylate, 2-Aminophenol, Kathon CG, Formaldehyde, 2-nitro-1,4-Phenylendiamine, 2,4-Dinitrochlorobenzoic acid, p-Phenylendiamine, 2-Mercaptobenzothiazole, Cinnamic alcohol and Resorcinol have replicates that group together, separate from the negative group. Still, as can be seen in FIG. 3C as well as in 3A, complete separation is not achieved with neither of the gene signatures of 1010 and 1137 genes respectively.

Backward Elimination Identifies Genes with the Most Discriminatory Power:

Even though the data set contains genes with p-values down to 1×10⁻¹⁷, lowering the p-value cutoff did not achieve complete separation between sensitizers and non-sensitizers. Gene signatures entirely selected on p-values does not provide the best possible predictive power, since a low p-value is no guarantee that a gene provides any additional information. To further reduce the number of transcripts for a predictive biomarker signature, we employed an algorithm for backward elimination (FIG. 4A). The algorithm removes genes one by one while taking into account not only the impact of genes individually, but how they perform collectively with the entire selected gene signature. For each gene eliminated, the Kullback-Leibler divergence (KLD) value is lowered, until a breakpoint is reached, at which point 200 genes remained. Continuing eliminating genes at this point causes the KLD to rise again, indicating that information is being lost (FIG. 4A). Therefore, the 200 genes with lowest KLD value were selected for further analysis. PCA of the 200 analytes revealed that they have the ability to completely separate sensitizers vs. non-sensitizers, indicating that these transcripts can be used as predictors for sensitizing properties of unknown samples (FIG. 4B). Importantly, by coloring the samples in the PCA by their potency, according to LLNA, it is clear that potency can be predicted by these genes, as well (FIG. 4C). The 200 genes are termed the “Prediction Signature” and their identity is listed in Table 3.

### Validation of the Analysis Method Used to Identify the Prediction Signature:

To validate the predictive power of our signature, we used a supervised learning method called the Support Vector Machine (SVM) [12], which maps the data from a training set in space in order to maximize the separation of gene expression induced by sensitizing and non-sensitizing chemicals. As training set, 70% of the data set was selected randomly and the entire selection process (as described above) was repeated. Starting with 29,141 transcripts, the signature was reduced to 200 transcripts, termed “Test Gene Signature”, using ANOVA filtering and backward elimination. The remaining 30% of the data set was used to test the signature obtained. The partitioning of the data set into subsets of 70% training data set and 30% test data set was done in a stratified random manner, meaning that the proportion of sensitizers and non-sensitizers in the complete data set are maintained in both the subsets, although the samples included in either of the two subsets are selected at random. Thereafter, the “Test Gene Signature” was used to train an SVM model with the training set, and the predictive power of the model was assessed with the test set. FIG. 5A shows a PCA plot based on the “Test Gene Signature” and the samples of the test set. Clearly, the separation between sensitizers (red) and non-sensitizers (green) resembles the one observed for the “Prediction Signature” in FIG. 4B. In the PCA of FIG. 5A, the samples of the sensitizing and non-sensitizing chemicals of the test set have been colored dark red and dark green respectively, indicating that they are not contributing to the principal components of the plot, but are merely plotted based on their expression values of the selected “Test Gene Signature”. As can be seen, sensitizers from the test set group with sensitizers from the training set, while non-sensitizers from the test set group with non-sensitizers from the training set. This is a very intuitive way of predicting the group belonging of the samples in the test set, using only the pattern recognition of the eye. The outcome of the SVM training and validation can be seen in FIG. 5B, where an area under the ROC curve of 1 confirms the ability of the “Test Gene Signature” to predict which group the sample belonged to in the test set. The prediction performance of the assay reveals an accuracy of 96%, sensitivity of 100% and specificity of 92%. While this experiment does not validate the prediction power of the “Prediction Signature” per se, it does indeed validate the method by which it has been selected, supporting the claim that the “Prediction Signature” is capable of accurately predict sensitizing properties of unknown samples.

### Molecular Functions and Canonical Pathways of the “Prediction Signature”.

Using Ingenuity Pathways Analysis (IPA, Ingenuity Systems Inc.), 184 of the 200 molecules in the signature were characterized with regard to functions and known (canonical) pathways. The remaining 16 molecules could not be mapped to any IPA entries. The dominating functions identified were small molecule biochemistry (38 molecules), cell death (33), lipid metabolism (24), hematological system development (19), cellular growth and proliferation (16), molecular transport (15), cell cycle (15) and carbohydrate metabolism (15), see table 4 for details. 67 of the 184 molecules were involved in the listed functions. Of the remaining 117 molecules, 30 were known from a variety of human diseases and molecular functions, such as described biomarkers (SCARB2, RFC2, VPS37A and BCL7A) and drug targets (ABAT). Most of these molecules are metabolic markers. In the signature as a whole, there are several drug targets, such as HMGCR, HMOX1, ABAT, RXRA, CD33, MAP2K1, MAPK13 and CD86. Two are described for skin disorders: CD86 (psoriasis) and RXRA (eczema). The signature also contains skin development (DHCR24) and dendritic cell markers (MAP2K1, NLRP12 and RFC2).

Pathways possibly invoked by the molecules in the signature were also investigated using IPA. Those most highly populated were NRF2 mediated oxidative response (10), xenobiotic metabolism signaling (8), LPS/IL-1 mediated inhibition of RXR function (6), aryl hydrocarbon receptor signaling (6) and protein kinase A signaling (6). The five highest ranked of these pathways are all known to take part in reactions provoked by foreign substances, xenobiotics. Xenobiotics are natural or synthetic chemical compounds, foreign to the human body.

### CONCLUSIONS

Allergic contact dermatitis (ACD) is an inflammatory skin disease caused by dysregulated adaptive immune responses to allergens [13]. Small molecular weight chemicals, so-called haptens, can bind self-proteins in the skin, which enables internalisation of the protein-bound allergenic chemical by skin dendritic cell (DC). DCs, under the influence of the local microenvironment, process the protein-hapten complex, migrate to the local lymph nodes and activate naïve T cells. The initiation and development of allergen-specific responses, mainly effector CD8+ T cells and Th1 cells, and production of immunoregulatory proteins, are hallmarks of the immune activation observed in ACD. This T-cell mediated type IV hypersensitivity reaction is characterised by symptoms such as rash, blisters and itching. ACD is the most common manifestation of immunotoxicity observed in humans [13] and hundreds of chemicals have been shown to cause sensitization in skin [14]. The driving factors and molecular mechanisms involved in sensitization are still unknown even though intense research efforts have been carried out to identify the immunological responses towards allergenic chemicals. The REACH legislation requires that all chemicals produced over 1 ton/year are tested for hazardous properties such as toxicity and allergenicity [5], which increase the demand for accurate assays for predictive power. Today, the identification of potential human sensitizers relies on animal experimentation, in particular the murine local lymph node assay (LLNA) [6]. The LLNA is based upon measurements of proliferation induced in draining lymph nodes of mice after chemical exposure [15]. Chemicals are defined as sensitizers if they provoke a three-fold increase in proliferation compared to control, and the amount of chemical required for the increase is the EC3 value. Thus, the LLNA can also be used to categorize the chemicals based on sensitisation potency. However, LLNA is in many ways not optimal. Besides the obvious ethical reasons, the assay is also time consuming and expensive. Human sensitization data often stem from human maximization tests (HMT) [16] and human patch tests (HPT). In an extensive report from the Interagency Coordinating Committee on the Validation of Alternative Methods (ICCVAM), the performance characteristics of LLNA were compared to other available animal-based methods and human sensitization data (HMT and HPT) [17]. The LLNA performance in comparison to human data (74 assessments) revealed an accuracy of 72%, a sensitivity of 72% and a specificity of 67%. Accuracy is defined as the proportion of correct outcomes of a method, sensitivity is the proportion of all positive chemicals that are correctly classified as positives, and specificity is the proportion of all negative chemicals that are correctly classified as negatives. Thus, there is a clear need to develop more reliable test methods for sensitization. Additionally, the 7th Amendment to the Cosmetics Directive (76/768/EEC) poses a complete ban on using animal experimentation for testing cosmetic ingredients by 2013 when a scientific reliable method is available. Thus, there is a great need from the industry for reliable predictive test methods that are based on human cells. Various human cell lines and primary cells involved in sensitization have been evaluated as predictive test system, such as epithelial cells, dendritic cells and T cells, however no validated test assay is currently available. Various single biomarkers have been suggested to be upregulated upon stimulation with sensitizing chemicals, such as CD40, CD80, CD54, CXCL8, IL-1 β, MIP-1β, p38 MAPK as reviewed in [18], yet singlehanded, none of them have the predictive power to discriminate between sensitizing and non-sensitizing chemicals. CD86 is among the markers most extensively studied; however, determining the expression level of this marker in our assay is relevant but not sufficient as readout for sensitization (FIG. 2). Only 9 out of 19 sensitizing chemicals induced a significant upregulation of CD86. Instead, it is our firm belief that the analyses of biomarker signatures are superior to any single biomarker. We therefore utilized the power of complete-genome transcriptomics and screened the gene regulation induced by a large set of well-defined chemicals and controls. The large number of differentially expressed genes in MUTZ-3 cells stimulated with sensitizing chemicals as compared to non-sensitizing controls, as identified with ANOVA (FIG. 3) revealed that MUTZ-3 indeed has a capacity to differentiate between these two groups, thus being a suitable cell system for in vitro assays for sensitization. Efforts have been made to create assays based on full genome analysis in various cell systems, such as CD34⁺-progenitor cells-derived DCs [19-21]. While such assays might provide in vivo like environments, one could argue that primary cells are not well suited for an assay of this great commercial interest. Moreover, the ethical aspect needs to be considered in such a system. Furthermore, previous efforts within in vitro assay development for sensitization that rely on full genome analysis have used a very limited set of testing compounds. To date, this study is the largest study performed within this area, utilizing 20 positive and 20 negative training compounds. Efforts have been made by us to divide these training compounds into two subsets, for training and testing respectively. While these experiments have resulted in successful predictions (data not shown), it is our experience that sensitizing compounds differ greatly in their induced gene expression profile, as can be seen in FIG. 3D. In this perspective, we wished to include as many training compounds as possible when identifying our “Prediction Signature”. Thus, we did not exclude any compounds for validation. Instead, we have validated the method by which the “Prediction Signature” was identified, by subdividing the samples into training and test sets at random, using unseen data for validation, as seen in FIG. 5. By including all sensitizing compounds, with a wide range of reactive mechanisms as well as sensitizing potencies, we argue that we have identified a prediction signature that is well suited for predicting sensitizing properties of unknown samples. Of important note, our “Prediction Signature” is able to predict the potency of sensitizing compounds, such as those defined by the LLNA, as is demonstrated in FIG. 4C. However, the potency predicted by LLNA and that of our classifier do not match for all samples. Notably, the moderate sensitizer 2-Hydroxyethyl acrylate show strong resemblance of strong and extreme sensitizers with respect to their gene expression profile. Similarly, the moderate sensitizers Ethylendiamine, Hexylcinnamic aldehyde, and Glyoxal group together with weak sensitizers. These findings provide vast implications that sensitizing potency, as defined, may need revising. We argue that our “Prediction Signature” may be used for such classifications.

In conclusion, we present an in vitro assay, based on a MUTZ-3 cell system that with an identified “Prediction Signature” consisting of 200 genes, which have the ability to correctly classify a sample as sensitizer or non-sensitizer. In addition, this assay can predict the potency of sensitizing compounds, and may be used to revise such classifications.

### Materials and Methods

### Chemicals

A panel of chemicals consisting of 20 sensitizers and 20 non-sensitizers were used for cell stimulations. The sensitizers were 2,4-Dinitrochlorobenzene, Cinnamaldehyde, Resorcinol, Oxazolone, Glyoxal, 2-Mercaptobenzothiazole, Eugenol, Isoeugenol, Cinnamic alcohol and p-Phenylendiamine, Formaldehyde, Ethylendiamine, 2-Hydroxyethyl acrylate, Hexylcinnamic aldehyde, Potassium Dichromate, Penicillin G, Katchon CG (MCI/MI), 2-aminophenol, Geraniol and 2-nitro-1,4-Phenylendiamine (Table 1). The non-sensitizers were Sodium dodecyl sulphate, Salicylic acid, Phenol, Glycerol, Lactic acid, Chlorobenzene, p-Hydrobenzoic acid, Benzaldehyde, Diethyl Phtalate and Octanoic acid, Zinc sulphate, 4-Aminobenzoic acid, Methyl salicylate, Ethyl vanillin, Isopropanol, Dimethyl formamide, 1-Butanol, Potassium permanganate, Propylene glycol and Tween 80 (Table 1). All chemicals were from Sigma-Aldrich, St. Louis, Mo., USA. Compounds were dissolved in either Dimethyl sulfoxide (DMSO) or distilled water. Prior to stimulations, the cytotoxicity of all compounds were monitored, using Propidium Iodide (PI) (BD Biosciences, San Diego, Calif.) using protocol provided by the manufacturer. The relative viability of stimulated cells was calculated as

$$Relative viability = \frac{fraction of viable stimulated cells}{fraction of viable unstimulated cells} \cdot 100$$

For toxic compounds, the concentration yielding 90% relative viability (Rv90) was used. For untoxic compounds, a concentration of 500 μM was used when possible. For non-toxic compounds that were insoluble at 500 μM in medium, the highest soluble concentration was used. For compounds dissolved in DMSO, the in-well concentration was 0.1% DMSO. The vehicle and concentrations used for each compound are listed in Table 2.

### Chemical Exposure of the Cells

The human myeloid leukaemia-derived cell line MUTZ-3 (DSMZ, Braunschweig, Germany) was maintained in α-MEM (Thermo Scientific Hyclone, Logan, Utah) supplemented with 20% (volume/volume) fetal calf serum (Invitrogen, Carlsbad, Calif.) and 40 ng/ml rhGM-CSF (Bayer HealthCare Pharmaceuticals, Seattle, Wash.), as described [10]. Prior to each experiment, the cells were immunophenotyped using flow cytometry as a quality control. Cells were seeded in 6-well plates at 200.000 cells/ml. Stock solutions of each compound was prepared in either DMSO or distilled water, and were subsequently diluted so the in-well concentrations corresponded to the Rv90 value, and in-well concentrations of DMSO were 0.1%. Cells were incubated for 24 h at 37° C. and 5% CO₂. Thereafter, cells were harvested and analysed with flow cytometry. In parallel, harvested cells were lysed in TRIzol reagent (Invitrogen) and stored at −20° C. until RNA extraction. Stimulations with chemicals were performed in three individual experiments, so that triplicates samples were obtained.

Phenotypic Analysis with Flow Cytometry

All cell surface staining and washing steps was performed in PBS containing 1% BSA (w/v). Cells were incubated with specific mouse mAbs for 15 min at 4° C. The following mAbs were used for flow cytometry: FITC-conjugated CD1a (DakoCytomation, Glostrup, Denmark), CD34, CD86, and HLA-DR (BD Biosciences), PE-conjugated CD14 (DakoCytomation), CD54 and CD80 (BD Biosciences). Mouse IgG1, conjugated to FITC or PE were used as isotype controls and PI was used to assess cell viability (BD Biosciences). FACSDiva software was used for data acquisition with FACSCanto II instrument (BD Bioscience). 10,000 events were acquired and gates were set based on light scatter properties to exclude debris and nonviable cells. Further data analysis was performed using FCS Express V3 (De Novo Software, Los Angeles, Calif.).

Preparation of cRNA and Gene Chip Hybridization

RNA isolation and gene chip hybridization was performed as described [22]. Briefly, RNA from unstimulated and chemical-stimulated MUTZ-3 cells, from triplicate experiments, were extracted and analysed. The preparation of labeled sense DNA was performed according to Affymetrix GeneChip Whole Transcript (WT) Sense Target Labeling Assay (100 ng Total RNA Labeling Protocol) using the recommended kits and controls (Affymetrix, Santa Clara, Calif.). Hybridization, washing and scanning of the Human Gene 1.0 ST Arrays were performed according to the manufacturer's protocol (Affymetrix).

### Microarray Data Analysis and Statistical Methods

The microarray data were normalised and quality checked with the RMA algorithm, using Affymetrix Expression Console (Affymetrix). Genes that were significantly regulated when comparing sensitizers with non-sensitizers were identified using one-way ANOVA, with false discovery rate (FDR) as a correction for multiple hypothesis testing. In order to reduce the large number of identified significant gene, we applied an algorithm developed in-house for Backward Elimination of analytes (Carlsson et al, unpublished). With this method, we train and test a Support Vector Machine (SVM) model [12] with leave-one out cross-validation, with one analyte left out. This process is iterated until each analyte has been left out once. For each iteration, a Kullback-Leibler divergence (KLD) is recorded, yielding N KLDs, where N is the number of analytes. The analyte that was left out when the smallest KLD was observed is considered to provide the least information in the data set. Thus, this analyte is eliminated and the iterations proceed, this time with N−1 analytes. In this manner, the analytes are eliminated one by one until a panel of markers remain that have been selected based on each analyte's ability to discriminate between sensitizers and non-sensitizers. The script for Backwards Eliminations was programmed for R [23], with the additional package e1071 [24]. ANOVA analyses and visualisation of results were performed in Qlucore Omics Explorer 2.1 (Qlucore, Lund, Sweden). The selected biomarker profile of 200 transcripts were designated the “Prediction Signature”.

### Validation of the Method for Identification of the “Prediction Signature”

In the absence of an external test data set, the data set was divided into a training set of 70% and a test set of 30% of the samples. The division was performed randomly, while maintaining the proportions of sensitizers and non-sensitizers in each subset at the same ratio as in the complete data set. A biomarker signature was identified in the training set using ANOVA filtering and Backward Elimination, as described above. This test signature was used to train an SVM, using the training set, which was thereafter applied to predict the samples of the test set. The distribution of the area under the Receiver Operating Characteristic (ROC) curve [25] was used as a measurement of the performance of the model.

### Assessment of Biological Functions of “Prediction Signature” Using Pathway Analysis

In order to investigate the biological functions the gene profile was analyzed using the Ingenuity Pathway Analysis software, IPA, (Ingenuity Systems, Inc. Mountain View, USA). The 200 top genes resulting from Backward Elimination were analyzed using the ‘build’ and ‘Path Explorer’ functions to build an interactome of the core genes from the “Prediction Signature” and connecting molecules suggested by IPA. The 200 molecules in the “Prediction Signature” were connected using the shortest known paths. In this process only human evidence from primary cells, cell lines and epidermal tissue was used. All molecules except for endogenous and chemical drugs were allowed in the network and all kinds of connections were allowed. Known ‘Functions’ and ‘Canonical Pathways’ from IPA were mapped to the interactome using the ‘Overlay’ function.

### ABBREVIATIONS

ACD, atopic contact dermatitis;
AML, acute myeloid leukemia cell;

### APC, Antigen Presenting Cell;

### DC, Dendritic Cell;

GM-CSF, Granulocyte macrophage colony-stimulating factor;
GPMT, Guinea pig maximization test;

### HMT, Human Maximation Test;

### HPTA, Human Patch Test Allergen;

### IL, Interleukin;

### LLNA, Local Lymph Node Assay;

### PCA, Principal Component Analysis

### REFERENCES

1. Akhavan, A. and S. R. Cohen, The relationship between atopic dermatitis and contact dermatitis. Clin Dermatol, 2003. 21(2): p. 158-62.
2. Mortz, C. G., et al., Prevalence of atopic dermatitis, asthma, allergic rhinitis, and hand and contact dermatitis in adolescents. The Odense Adolescence Cohort Study on Atopic Diseases and Dermatitis. Br J Dermatol, 2001. 144(3): p. 523-32.
3. Nielsen, N. H., et al., Allergic contact sensitization in an adult Danish population: two cross-sectional surveys eight years apart (the Copenhagen Allergy Study). Acta Derm Venereol, 2001. 81(1): p. 31-4.
4. Fonacier, L. S., S. C. Dreskin, and D. Y. Leung, Allergic skin diseases. J Allergy Clin Immunol. 125(2 Suppl 2): p. S138-49.
5. EC 1907/2006, in Regulation (EC) No 1907/2006 of the European Parliament and of the Council of 18 Dec. 2006 concerning the Registration, Evaluation, Authorisation and Restriction of Chemicals (REACH), establishing a European Chemicals Agency, amending Directive 1999/45/EC and repealing Council Regulation (EEC) No 793/93 and Commission Regulation (EC) No 1488/94 as well as Council Directive 76/769/EEC and Commission Directives 91/155/EEC, 93/67/EEC, 93/105/EC and 2000/21/EC.
6. Basketter, D. A., et al., Local lymph node assay—validation, conduct and use in practice. Food Chem Toxicol, 2002. 40(5): p. 593-8.
7. Magnusson, B. and A. M. Kligman, The identification of contact allergens by animal assay. The guinea pig maximization test. J Invest Dermatol, 1969. 52(3): p. 268-76.
8. Masterson, A. J., et al., MUTZ-3, a human cell line model for the cytokine-induced differentiation of dendritic cells from CD34+ precursors. Blood, 2002. 100(2): p. 701-3.
9. Santegoets, S. J., et al., A CD34(+) human cell line model of myeloid dendritic cell differentiation: evidence for a CD14(+)CD11b(+) Langerhans cell precursor. J Leukoc Biol, 2006. 80(6): p. 1337-44.
10. Larsson, K., M. Lindstedt, and C. A. Borrebaeck, Functional and transcriptional profiling of MUTZ-3, a myeloid cell line acting as a model for dendritic cells. Immunology, 2006. 117(2): p. 156-66.
11. Rasaiyaah, J., et al., Dendritic cells and myeloid leukaemias: plasticity and commitment in cell differentiation. Br J Haematol, 2007. 138(3): p. 281-90.
12. Cortes, C. and V. Vapnik, Support-Vector Networks. Machine Learning, 1995. 20(3): p. 273-297.
13. Kimber, I., et al., Allergic contact dermatitis. Int Immunopharmacol, 2002. 2(2-3): p. 201-11.
14. Gerberick, G. F., et al., Compilation of historical local lymph node data for evaluation of skin sensitization alternative methods. Dermatitis, 2005. 16(4): p. 157-202.
15. Kimber, I., et al., Classification of contact allergens according to potency: proposals. Food Chem Toxicol, 2003. 41(12): p. 1799-809.
16. Kligman, A. M., The identification of contact allergens by human assay. 3. The maximization test: a procedure for screening and rating contact sensitizers. J Invest Dermatol, 1966. 47(5): p. 393-409.
17. Haneke, K. E., et al., ICCVAM evaluation of the murine local lymph node assay. Data analyses completed by the National Toxicology Program Interagency Center for the Evaluation of Alternative Toxicological Methods. Regul Toxicol Pharmacol, 2001. 34(3): p. 274-86.
18. dos Santos, G. G., et al., Progress on the development of human in vitro dendritic cell based assays for assessment of the sensitizing potential of a compound. Toxicol Appl Pharmacol, 2009. 236(3): p. 372-82.
19. Hooyberghs, J., et al., A cell-based in vitro alternative to identify skin sensitizers by gene expression. Toxicol Appl Pharmacol, 2008. 231(1): p. 103-11.
20. Lambrechts, N., et al., Gene markers in dendritic cells unravel pieces of the skin sensitization puzzle. Toxicol Lett, 2010. 196(2): p. 95-103.
21. Lambrechts, N., et al., Assessment of chemical skin-sensitizing potency by an in vitro assay based on human dendritic cells. Toxicol Sci, 2010. 116(1): p. 122-9.
22. Lindstedt, M., et al., Genomic and functional delineation of dendritic cells and memory T cells derived from grass pollen-allergic patients and healthy individuals. Int Immunol, 2005. 17(4): p. 401-9.
23. R Development Core Team. R: A language and environment for statistical computing. R Foundation for Statistical Computing. 2008; Available from: http://www.R-project.org.
24. R package e1071. Available from: http://cran.r-project.org/web/packages/e1071/index.html.
25. Lasko, T. A., et al., The use of receiver operating characteristic curves in biomedical informatics. J Biomed Inform, 2005. 38(5): p. 404-15.
26. Warbrick, E. V., et al., Influence of application vehicle on skin sensitization to methylchloroisothiazolinone/methylisothiazolinone: an analysis using the local lymph node assay. Contact Dermatitis, 1999. 41(6): p. 325-9.
27. Roberts, D. W., et al., Mechanistic applicability domain classification of a local lymph node assay dataset for skin sensitization. Chem Res Toxicol, 2007. 20(7): p. 1019-30.
28. Gerberick, G. F., et al., Contact allergenic potency: correlation of human and local lymph node assay data. Am J Contact Dermat, 2001. 12(3): p. 156-61.
29. Basketter, D. A., D. Sanders, and I. R. Jowsey, The skin sensitization potential of resorcinol: experience with the local lymph node assay. Contact Dermatitis, 2007. 56(4): p. 196-200.
30. Ryan, C. A., et al., Activity of human contact allergens in the murine local lymph node assay. Contact Dermatitis, 2000. 43(2): p. 95-102.
31. APPENDIX C; Comparative LLNA: BrdU-FC, Traditional LLNA, Guinea Pig Skin Sensitization, and Human Data. 2008 Available from: http://iccvam.niehs.nih.gov/methods/immunotoxficLLNA/Appx/AppendixC_LL NA_FC07Jan08FD.pdf.
32. Patlewicz, G., et al., Skin-sensitization structure-activity relationships for aldehydes. Contact Dermatitis, 2001. 44(6): p. 331-6.
33. Basketter, D. A., G. F. Gerberick, and I. Kimber, Strategies for identifying false positive responses in predictive skin sensitization tests. Food Chem Toxicol, 1998. 36(4): p. 327-33.
34. Ashby, J., et al., Structure activity relationships in skin sensitization using the murine local lymph node assay. Toxicology, 1995. 103(3): p. 177-94.
35. Sakaguchi, H., et al., Development of an in vitro skin sensitization test using human cell lines; human Cell Line Activation Test (h-CLAT). II. An inter-laboratory study of the h-CLAT. Toxicol In Vitro, 2006. 20(5): p. 774-84.
36. The Murine Local Lymph Node Assay: A Test Method for Assessing the Allergic Contact Dermatitis Potential of Chemicals/Compounds. 1999; Available from: http://iccvam.niehs.nih.gov/docs/immunotox_docs/llna/llnarep.pdf.

### Tables

TABLE 1
List of sensitizing and non-sensitizing chemicals, based on murine LLNA
classification, tested in the cell-based assay.

| Compound | Abbreviation | Potency | LLNA | HMT¹ | HPTA¹ |
|---|---|---|---|---|---|
| Sensitizers | | | | | |
| 2,4-Dinitrochlorobenzene | DNCB | Extreme [15] | + [15] | | |
| Oxazolone | OM | Extreme [15] | + [15] | | |
| Potassium dichromate | PD | Extreme [14] | + [14] | + | + |
| Kathon CH (MC/MCI) | KCG | Extreme [14, 26] | + [14, 26] | | |
| Formaldehyde | FA | Strong [15] | + [15] | + | + |
| 2-Aminophenol | 2AP | Strong [27] | + [27] | | |
| 2-nitro-1,4-Phenylendiamine | NPDA | Strong [27] | + [27] | | |
| p-Phenylendiamine | PPD | Strong [28] | + [28] | + | + |
| Hexylcinnamic aldehyde | HCA | Moderate [15] | + [15] | | |
| 2-Hydroxyethyl acrylate | 2HA | Moderate [27] | + [27] | | + |
| 2-Mercaptobenzothiazole | MBT | Moderate [27] | + [27] | + | + |
| Glyoxal | GO | Moderate [27] | + [27] | + | |
| Cinnamaldehyde | CALD | Moderate [28] | + [28] | + | + |
| Isoeugenol | IEU | Moderate [28] | + [28] | | + |
| Ethylendiamine | EDA | Moderate [14] | + [14] | | |
| Resorcinol | RC | Moderate [29] | + [29] | − | + |
| Cinnamic alcohol | CALC | Weak [27] | + [28] | | |
| Eugenol | EU | Weak [28] | + [28] | | + |
| Penicillin G | PEN G | Weak [28] | + [28] | + | |
| Geraniol | GER | Weak [14] | + [14] | − | + |
| Non-sensitizers | | | | | |
| 1-Butanol | BUT | | − [30] | | |
| 4-Aminobenzoic acid | PABA | | − [31] | − | + |
| Benzaldehyde | BA | | − [32] | | |
| Chlorobenzene | CB | | − [14] | | |
| Diethyl phthalate | DP | | − [28] | | |
| Dimethyl formamide | DF | | − [26] | | |
| Ethyl vanillin | EV | | − [32] | | |
| Glycerol | GLY | | − [28] | | |
| Isopropanol | IP | | − [28] | | |
| Lactic acid | LA | | − [14] | | |
| Methyl salicylate | MS | | − [14] | − | |
| Octanoic acid | OA | | − [33] | | |
| Propylene glycol | PG | | − [31] | | |
| Phenol | PHE | | − [33] | − | |
| p-Hydroxybenzoic acid | HBA | | − [34] | | |
| Potassium permanganate | PP | | − | | |
| Salicylic acid | SA | | − [14] | − | |
| Sodium dodecyl sulphate | SDS | | +² [14, 33] | − | |
| Tween 80 | T80 | | − [35] | | + |
| Zinc sulphate | ZS | | +² [36] | | |

¹HMT, Human Maximation Test; HPTA, Human Patch Test Allergen. Information is derived from [17].
²False positives in LLNA.

TABLE 2
Vehicle and concentrations used for testing.

| | | | Max | | |
|---|---|---|---|---|---|
| | | | solubility | Rv90 | Concentration |
| Compound | Abbreviation | Vehicle | (μM) | (μM) | In culture (μM) |
| Sensitizers | | | | | |
| 2,4-Dinitrochlorobenzene | DNCB | DMSO | — | 4 | 4 |
| Oxazolone | OXA | DMSO | 250 | — | 250 |
| Potassium dichromate | PD | Water | 51.02 | 1.5 | 1.5 |
| Kathon CG (MC/MCI)* | KCG | Water | — | 0.0035% | 0.0035% |
| Formaldehyde | FA | Water | — | 80 | 80 |
| 2-Aminophenol | 2AP | DMSO | — | 100 | 100 |
| 2-nitro-1,4-Phenylendiamine | NPDA | DMSO | — | 300 | 300 |
| p-Phenylendiamine | PPD | DMSO | 566 | 75 | 75 |
| Hexylcinnamic aldehyde | HCA | DMSO | 32.34 | — | 32.24 |
| 2-Hydroxyethyl acrylate | 2HA | Water | — | 100 | 100 |
| 2-Mercaptobenzothiazole | MBT | DMSO | 250 | — | 250 |
| Glyoxal | GO | Water | — | 300 | 300 |
| Cinnamaldehyde | CALD | Water | — | 120 | 120 |
| Isoeugenol | IEU | DMSO | 641 | 300 | 300 |
| Ethylendiamine | EDA | Water | — | — | 500 |
| Resorcinol | RC | Water | — | — | 500 |
| Cinnamic alcohol | CALC | DMSO | 500 | — | 500 |
| Eugenol | EU | DMSO | 649 | 300 | 300 |
| Penicillin G | PEN G | Water | — | — | 500 |
| Geraniol | GER | DMSO | — | — | 500 |
| Non-sensitizers | | | | | |
| 1-Butanol | BUT | DMSO | — | — | 500 |
| 4-Aminobenzoic acid | PABA | DMSO | — | — | 500 |
| Benzaldehyde | BA | DMSO | 250 | — | 250 |
| Chlorobenzene | CB | DMSO | 98 | — | 98 |
| Diethyl phthalate | DP | DMSO | 50 | — | 50 |
| Dimethyl formamide | DF | Water | — | — | 500 |
| Ethyl vanillin | EV | DMSO | — | — | 500 |
| Glycerol | GLY | Water | — | — | 500 |
| Isopropanol | IP | Water | — | — | 500 |
| Lactic acid | LA | Water | — | — | 500 |
| Methyl salicylate | MS | DMSO | — | — | 500 |
| Octanoic acid | OA | DMSO | 504 | — | 500 |
| Peopylene glycol | PG | Water | — | — | 500 |
| Phenol | PHE | Water | — | — | 500 |
| p-Hydroxybenzoic acid | HBA | DMSO | 250 | — | 250 |
| Potassium permanganate | PP | Water | 38 | — | 38 |
| Salicylic acid | SA | DMSO | — | — | 500 |
| Sodium dodecyl sulphate | SDS | Water | — | 200 | 200 |
| Tween 80 | T80 | DMSO | — | — | 500 |
| Zinc sulphate | ZS | Water | 126 | — | 126 |

¹Kathon CG is a mixture of the compounds MC and MCI. The concentration of this mixture is given in %.

TABLE 3
Differentially expressed genes in MUTZ-3 cells stimulated with sensitizing
chemicals as compared to non-sensitizing agents and controls.

| | | |
|---|---|---|
| | | NCBI reference |
| Gene Title | Gene Symbol | sequence |

Table 3A

| | | |
|---|---|---|
| fatty acid synthase | FASN | NM_004104 |
| squalene epoxidase | SQLE | NM_003129 |
| taste receptor, type 2, member 5 | TAS2R5 | NM_018980 |
| keratinocyte growth factor-like protein 1/2/hypothetical protein | KGFLP1/2/FLJ20444 | AF523265 |
| FLJ20444 | | |
| transmembrane anterior posterior transformation 1 | TAPT1 | NM_153365 |
| Sprouty homolog 2 | SPRY2 | NM_005842 |
| B-cell CLL/lymphoma 7A | BCL7A | NM_020993 |
| solute carrier family 25, member 32 | SLC25A32 | NM_030780 |
| ferritin, heavy polypeptide pseudogene 1 | FTHP1 | GENSCAN00000008165 |
| ATPase, H+ transporting, lysosomal 50/57 kDa, V1 subunit H | ATP6V1H | NM_015941 |
| Histone cluster 1, H1e | HIST1H1E | NM_005321 |

Table 3B

| | | |
|---|---|---|
| 4-aminobutyrate aminotransferase | ABAT | NM_020686 |
| abhydrolase domain containing 5 | ABHD5 | NM_016006 |
| alkaline ceramidase 2 | ACER2 | NM_001010887 |
| ATP citrate lyase | ACLY | NM_001096 |
| actin-related protein 10 homolog | ACTR10 | NM_018477 |
| ADAM metallopeptidase domain 20 | ADAM20 | NM_003814 |
| Retrotransposed pseudogene AL391261.2-201 | AL391261.2-201 | GENSCAN00000063078 |
| aldehyde dehydrogenase 18 family, member A1 | ALDH18A1 | NM_002860 |
| aldehyde dehydrogenase 1 family, member B1 | ALDH1B1 | NM_000692 |
| alkB, alkylation repair homolog 6 (E. coli) | ALKBH6 | NM_032878 |
| anaphase promoting complex subunit 1 | ANAPC1 | NM_022662 |
| anaphase promoting complex subunit 5 | ANAPC5 | NM_016237 |
| ankyrin repeat, family A (RFXANK-like), 2 | ANKRA2 | NM_023039 |
| ADP-ribosylation factor GTPase activating protein 3 | ARFGAP3 | NM_014570 |
| Rho GTPase activating protein 9 | ARHGAP9 | NM_032496 |
| ankyrin repeat and SOLS box-containing 7 | ASB7 | NM_198243 |
| ATPase, H+ transporting, lysosomal 9 kDa, V0 subunit e1 | ATP6V0E1 | NM_003945 |
| bridging integrator 2 | BIN2 | NM_016293 |
| bleomycin hydrolase | BLMH | NM_000386 |
| brix domain containing 1/ribosome production factor 2 | BXDC1/RPF2 | ENST00000368864 |
| homolog | | |
| chromosome 11 open reading frame 61 | C11orf61 | NM_024631 |
| chromosome 11 open reading frame 67 | C11orf67 | NM_024684 |
| chromosome 12 open reading frame 57 | C12orf57 | NM_138425 |
| chromosome 13 open reading frame 18 | C13orf18 | NM_025113 |
| chromosome 15 open reading frame 24 | C15orf24 | NM_020154 |
| chromosome 19 open reading frame 54 | C19orf54 | NM_198476 |
| chromosome 1 open reading frame 174 | C1orf174 | NM_207356 |
| chromosome 1 open reading frame 183 | C1orf183 | NM_019099 |
| chromosome 20 open reading frame 111 | C20orf111 | NM_016470 |
| chromosome 20 open reading frame 24 | C20orf24 | BC004446 |
| chromosome 3 open reading frame 62/ubiquitin specific | C3orf62/USP4 | BC023586 |
| peptidase 4 (proto-oncogene) | | |
| chromosome 9 open reading frame 89 | C9orf89 | BC038856 |
| coactivator-associated arginine methyltransferase 1 | CARM1 | NM_199141 |
| CD33 molecule | CD33 | NM_001772 |
| CD86 molecule | CD86 | NM_175862 |
| CD93 molecule | CD93 | NM_012072 |
| cytochrome c oxidase subunit VIIa polypeptide 2 like | COX7A2L | NM_004718 |
| corticotropin releasing hormone binding protein | CRHBP | NM_001882 |
| chondroitin sulfate N-acetylgalactosaminyltransferase 2 | CSGALNACT2 | NM_018590 |
| Cytochrome P450 51A1 | CYP51A1 | NM_000786.2 |
| DDRGK domain containing 1 | DDRGK1 | NM_023935 |
| DEAD (Asp-Glu-Ala-Asp) box polypeptide 21 | DDX21 | NM_004728 |
| 24-dehydrocholesterol reductase | DHCR24 | NM_014762 |
| 7-dehydrocholesterol reductase | DHCR7 | NM_001360 |
| DEAR (Asp-Glu-Ala-His) box polypeptide 33 | DHX33 | NM_020162 |
| DnaJ (Hsp40) homolog, subfamily B, member 4 | DNAJB4 | NM_007034 |
| DnaJ (Hsp40) homolog, subfamily B, member 9 | DNAJB9 | NM_012328 |
| DnaJ (Hsp40) homolog, subfamily C, member 5 | DNAJC5 | NM_025219 |
| DnaJ (Hsp40) homolog, subfamily C, member 9 | DNAJC9 | NM_015190 |
| D-tyrosyl-tRNA deacylase 1 homolog | DTD1 | NM_080820 |
| ER degradation enhancer, mannosidase alpha-like 2 | EDEM2 | NM_018217 |
| ecotropic viral integration site 2B | EVI2B | NM_006495 |
| family with sequence similarity 36, member A | FAM36A | NM_198076 |
| family with sequence similarity 86, member A | FAM86A | NM_201400 |
| Fas (TNF receptor superfamily, member 6) | FAS | NM_000043 |
| MGC44478 | FDPSL2A | NR_003262 |
| ferredoxin reductase | FDXR | NM_024417 |
| forkhead box O4 | FOXO4 | NM_005938 |
| FTHL10-001, Transcribed processed pseudogene | FTHL10-001 | NR_002200 |
| fucosidase, alpha-L-2, plasma | FUCA2 | NM_032020 |
| growth arrest-specific 2 like 3 | GAS2L3 | NM_174942 |
| ganglioside induced differentiation associated protein 2 | GDAP2 | NM_017686 |
| growth differentiation factor 11 | GDF11 | NM_005811 |
| glutaredoxin (thioltransferase) | GLRX | NM_002064 |
| guanine nucleotide binding protein-like 3 | GNL3L | NM_019067 |
| glucosamine-phosphate N-acetyltransferase 1 | GNPNAT1 | NM_198066 |
| glutathione reductase | GSR | NM_000637 |
| GTF2I repeat domain containing 2B/2/2 pseudogene | GTF2IRD2B/2/2P | BC067859 |
| general transcription factor IIIC, polypeptide 2 beta | GTF3C2 | NM_001521 |
| HMG-box transcription factor 1 | HBP1 | NM_012257 |
| histone cluster 1, H1c | HIST1H1C | NM_005319 |
| histone cluster 1, H2ae | HIST1H2AE | NM_021052 |
| histone cluster 1, H2be | HIST1H2BE | NM_003523 |
| histone clusters 1, H2bm/2, H3, pseudogene 2/2, H2b/a | HIST1H2BM/ | NM_001024599 |
| | HIST2H3PS2/BF/A | |
| histone cluster 1, H3g | HIST1H3G | NM_003534 |
| histone cluster 1, H3j | HIST1H3J | NM_003535 |
| histone cluster 1, H4a | HIST1H4A | NM_003538 |
| histone clusters 2, H2aa3/2, H2aa4 | HIST2H2AA3/4 | NM_003516 |
| high-mobility group box 3 | HMGB3 | NM_005342 |
| 3-hydroxy-3-methylglutaryl-Coenzyme A reductase | HMGCR | NM_000859 |
| 3-hydroxy-3-methylglutaryl-Coenzyme A synthase 1 | HMGCS1 | NM_001098272 |
| heme oxygenase (decycling) 1 | HMOX1 | NM_002133 |
| heterogeneous nuclear ribonucleoprotein L | HNRNPL | NM_001533 |
| insulin receptor substrate 2 | IRS2 | NM_003749 |
| iron-sulfur cluster scaffold homolog | ISCU | NM_014301 |
| interferon stimulated exonuclease gene 20 kDa-like 2 | ISG20L2 | NM_030980 |
| potassium voltage-gated channel, Isk-related family, member 3 | KCNE3 | NM_005472 |
| hypothetical protein LOC100132855/ATPase, H+ | LOC100132855/ | NM_004691 |
| transporting, lysosomal 38 kDa, V0 subunit d1 | ATP6V0D1 | |
| hCG1651476 | LOC284417 | NM_001085488 |
| golgi autoantigen, golgin subfamily a, 6 pseudogene/ | LOC729668/MTPAP | NM_018109 |
| mitochondrial poly(A) polymerase | | |
| lysophosphatidic acid receptor 1 | LPAR1 | NM_057159 |
| leucine-rich PPR-motif containing | LRPPRC | NM_133259 |
| lymphocyte antigen 96 | LY96 | NM_015364 |
| mitogen-activated protein kinase kinase 1 | MAP2K1 | NM_002755 |
| mitogen-activated protein kinase 13 | MAPK13 | NM_002754 |
| methyltransferase like 2A | METTL2A | NM_181725 |
| Brain cDNA clone: similar to human METTL2 | METTL2B | NM_018396.1 |
| Methyltransferase like 2B | METTL2B | NM_018396.2 |
| microsomal glutathione S-transferase 3 | MGST3 | NM_004528 |
| mitochondrial ribosomal protein L30 | MRPL30 | NM_145212 |
| mitochondrial ribosomal protein L4 | MRPL4 | NM_146388 |
| mitochondrial ribosomal protein S17 | MRPS17 | NM_015969 |
| 5-methyltetrahydrofolate-homocysteine methyltransferase | MTR | NM_000254 |
| MYB binding protein (P160) 1a | MYBBP1A | NM_014520 |
| neighbor of BRCA1 gene 1 | NBR1 | NM_031858 |
| nuclear import 7 homolog | NIP7 | NM_016101 |
| NLR family, pyrin domain containing 12 | NLRP12 | NM_144687 |
| nucleolar protein family 6 (RNA-associated) | NOL6 | NM_022917 |
| NAD(P)H dehydrogenase, quinone 1 | NQO1 | NM_000903 |
| nuclear receptor binding protein 1 | NRBP1 | NM_013392 |
| nucleotide binding protein-like | NUBPL | NM_025152 |
| nudix (nucleoside diphosphate linked moiety X)-type motif 14 | NUDT14 | NM_177533 |
| nuclear fragile X mental retardation protein interacting protein 1 | NUFIP1 | NM_012345 |
| nucleoporin 153 kDa | NUP153 | NM_005124 |
| olfactory receptor, family 5, subfamily B, member 21 | OR5B21 | NM_001005218 |
| PAS domain containing serine/threonine kinase | PASK | NM_015148 |
| PRKC, apoptosis, WT1, regulator | PAWR | NM_002583 |
| PDGFA associated protein 1 | PDAP1 | NM_014891 |
| phosphodiesterase 1B, calmodulin-dependent | PDE1B | NM_000924 |
| phosphoribosylformylglycinamidine synthase | PFAS | NM_012393 |
| pleckstrin homology-like domain, family A, member 3 | PHLDA3 | NM_012396 |
| phosphoinositide-3-kinase adaptor protein 1 | PIK3AP1 | NM_152309 |
| PTEN induced putative kinase 1 | PINK1 | NM_032409 |
| phosphomannomutase 2 | PMM2 | NM_000303 |
| partner of NOB1 homolog | PNO1 | NM_020143 |
| polymerase (RNA) II (DNA directed) polypeptide E, 25 kDa | POLR2E | NM_002695 |
| polymerase (RNA) III (DNA directed) polypeptide E (80 kD) | POLR3E | NM_018119 |
| protein phosphatase 1D magnesium-dependent, delta isoform | PPM1D | BC042418 |
| phosphatidylinositol-3,4,5-trisphosphate-dependent Rac | PREX1 | NM_020820 |
| exchange factor 1 | | |
| proline-serine-threonine phosphatase interacting protein 1 | PSTPIP1 | NM_003978 |
| prothymosin, alpha | PTMA | NM_016184/024809 |
| RAB33B, member RAS oncogene family | RAB33B | NM_031296 |
| renin binding protein | RENBP | NM_002910 |
| replication factor C (activator 1) 2, 40 kDa | RFC2 | NM_181471 |
| ribonuclease H1 | RNASEH1 | NM_002936 |
| ring finger protein 146 | RNF146 | NM_030963 |
| ring finger protein 24 | RNF24 | NM_007219 |
| ring finger protein 26 | RNF26 | NM_032015 |
| Havana pseudogene RP1-274L14.2-001 | RP1-274L14.2-001 | NM_032020 |
| ribosomal protein SA/small nucleolar RNA, H/ACA box 62 | RPSA/SNORA62 | NM_014570 |
| RNA pseudouridylate synthase domain containing 2 | RPUSD2 | NM_152260 |
| ribosomal RNA processing 12 homolog | RRP12 | NM_015179 |
| retinoid X receptor, alpha | RXRA | NM_002957 |
| scavenger receptor class B, member 2 | SCARB2 | NM_005506 |
| SERPINE1 mRNA binding protein 1 | SERBP1 | NM_001018067 |
| splicing factor proline/glutamine-rich | SFPQ | NM_005066 |
| solute carrier family 35, member B3 | SLC35B3 | BX538271 |
| solute carrier family 37, member 4 | SLC37A4 | NM_001467 |
| solute carrier family 5, member 6 | SLC5A6 | NM_021095 |
| sphingomyelin phosphodiesterase 4, neutral membrane | SMPD4 | NM_017751 |
| small nucleolar(sn)RNA host gene 1, non-coding/snRNA C/D | SNHG1/SNORD26 | NM_002032 |
| box 26 | | |
| small nucleolar RNA host gene 12 (non-coding) | SNHG12 | NM_207356 |
| small nucleolar RNA, H/ACA box 45 | SNORA45 | NR_002977 |
| SnRNA | SnRNA | Affymetrix: 7966223 |
| sorting nexin family member 27 | SNX27 | NM_030918 |
| sterol regulatory element binding transcription factor 2 | SREBF2 | NM_004599 |
| RRNA SSU_rRNA_5 | SSU_rRNA_5 | ENST00000386723 |
| ST3 beta-galactoside alpha-2,3-sialyltransferase 6 | ST3GAL6 | NM_006100 |
| serine/threonine kinase 17b | STK17B | NM_004226 |
| tubulin folding cofactor E-like | TBCEL | NM_152715 |
| tectonic family member 2 | TCTN2 | NM_024809 |
| toll-like receptor 6 | TLR6 | NM_006068 |
| toll-like receptor 9/twinfilin homolog 2 | TLR9/TWF2 | NM_007284 |
| transmembrane protein 55A | TMEM55A | NM_018710 |
| transmembrane protein 59 | TMEM59 | NM_004872 |
| transmembrane protein 77 | TMEM77 | BC091509 |
| transmembrane protein 97 | TMEM97 | NM_014573 |
| tumor necrosis factor receptor superfamily, member 10c | TNFRSF10C | NM_003841 |
| translocase of outer mitochondrial membrane 34 | TOMM34 | NM_006809 |
| translocase of outer mitochondrial membrane 40 homolog | TOMM40 | BC001779 |
| translocase of outer mitochondrial membrane 5 homolog/F- | TOMM5/FBX010 | NM_012166 |
| box protein 10 | | |
| tumor protein p53 inducible protein 3 | TP53I3 | NM_004881 |
| tumor protein p53 inducible nuclear protein 1 | TP53INP1 | NM_033285 |
| thioredoxin reductase 1 | TXNRD1 | NM_003330 |
| ubiquitin-fold modifier conjugating enzyme 1 | UFC1 | NM_016406 |
| ubiquitin specific peptidase 10 | USP10 | NM_005153 |
| vesicle-associated membrane protein 3 (cellubrevin) | VAMP3 | NM_004781 |
| valyl-tRNA synthetase | VARS | NM_006295 |
| vacuolar protein sorting 37 homolog A | VPS37A | NM_152415 |
| zinc finger protein 211 | ZNF211 | NM_006385 |
| zinc finger protein 223 | ZNF223 | NM_013361 |
| zinc finger protein 561 | ZNF561 | NM_152289 |
| zinc finger protein 79 | ZNF79 | NM_007135 |

Table 3 legend.
The table shows the profile genes found by t-test and Backward Elimination. Genes were annotated, using the NetAffx database from Affymetrix (www.affymetrix.com, Santa Clara USA). When found, the Unigene (www.ncbi.nlm.nih.gov/UniGene/) ID was chosen as the gene identifier. In the twelve cases where no Unigene ID was reported the best alternative ID was given. Gene names and IDs were checked against the IPA database where 189 of the 200 could be matched. In one instance only an Affymetrix ID was reported. 6 duplicate genes were removed.

TABLE 4
Dominating functions in the “Prediction signature”. 184 of the 200 molecules
were investigated functionally using IPA. Only functions populated by 15 or more
molecules were reported

| | Number of | | |
|---|---|---|---|
| | molecules | | |
| | from | | Most prominent |
| Function | signature | Molecule names | sub functions |
| small molecule | 38 | ABHD5, ACLY, ALDH18A1, BLMH, CD86, | Metabolism (23), |
| biochemistry | | CSGALNACT2, CYP51A1, DHCR24, DHCR7, DNAJC5, | biosynthesis (14), |
| | | FAS, FASN, FDXR, GLRX, GNPNAT1, HMGCR, | modification (12), |
| | | HMOX1, IRS2, LPAR1, LY96, MGST3, MTR, NQO1, | synthesis (10) |
| | | PASK, PDE1B, PINK1, PMM2, RENBP, RXRA, | |
| | | SLC25A32, SLC37A4, SLC5A6, SMPD4, SQLE, | |
| | | SREBF2, ST3GAL6, TLR6, TMEM55A | |
| cell death | 33 | CD33, DDX19A, DHCR24, DNAJB9, DNAJC5, FAS, | Apoptosis (30), cell |
| | | FASN, FDXR, FOXO4, GLRX, GNPNAT1, GSR, | death (13) |
| | | HIST1H1C, HMGB3, HMOX1, IRS2, LPAR1, MAP2K1, | |
| | | MAPK13, NQO1, PAWR, PDE1B, PHLDA3, PINK1, | |
| | | PPM1D, RXRA, SERBP1, SPRY2, STK17B, TLR6, | |
| | | TNFRSF10C, TP53INP1, TXNRD1 | |
| lipid metabolism | 24 | ABHD5, ACLY, CYP51A1, DHCR24, DHCR7, FAS, | Metabolism (17), |
| | | FASN, FDXR, HMGCR, HMOX1, IRS2, LPAR1, LY96, | modification (11), |
| | | MGST3, PASK, RENBP, RXRA, SLC37A4, SMPD4, | synthesis (10) |
| | | SQLE, SREBF2, ST3GAL6, TLR6, TMEM55A | |
| hematological | 19 | CARM1, CD33, CD86, FAS, FOXO4, HIST1H1C, | Proliferation (10), |
| system | | HMGB3, HMGCR, HMOX1, IRS2, LY96, NBR1, NQO1, | apoptosis (5) |
| development | | PAWR, PIK3AP1, PPM1D, STK17B, TP53INP1, VAMP3 | |
| cellular growth | 16 | CARM1, CD33, CD86, FAS, FOXO4, HIST1H1C, | Proliferation (16), |
| and proliferation | | HMGB3, HMGCR, HMOX1, IRS2, LY96, NBR1, NQO1, | growth (4) |
| | | PAWR, PIK3AP1, PPM1D, STK17B, TP53INP1, VAMP3 | |
| molecular | 15 | CARM1, CD33, CD86, FAS, FOXO4, HIST1H1C, | Accumulation (8), |
| transport | | HMGB3, HMGCR, HMOX1, IRS2, LY96, NBR1, NQO1, | quantity (5) |
| | | PAWR, PIK3AP1, PPM1D, STK17B, TP53INP1, VAMP3 | |
| cell cycle | 15 | DNAJB4, DTD1, FAS, FASN, FOXO4, GDF11, HBP1, | Cell cycle |
| | | HMOX1, IRS2, MAP2K1, PAWR, PPM1D, SFPQ, | progression (13), |
| | | SPRY2, TP53INP1 | cell division (5) |
| carbohydrate | 15 | DNAJB4, DTD1, FAS, FASN, FOXO4, GDF11, HBP1, | Metabolism (9), |
| metabolism | | HMOX1, IRS2, MAP2K1, PAWR, PPM1D, SFPQ, | biosynthesis (5) |
| | | SPRY2, TP53INP1 | |

TABLE 5
Support Vector Machine (SVM) algorithm

```
filnamnTraining<−“training set.txt”
filnamnTest<−“test set.txt”
lista <− read.delim(“biomarker signature.txt”,header=FALSE) # EN FIL
MED DE ANALYTER
lista <− as.character(lista[[1]])
listaBoolean <− is.element(ProteinNames, lista)
group1<− “pos”
group2<− “neg”
rawfile <− read.delim(filnamnTraining)
samplenames <− as.character(rawfile[,1])
groupsTraining <− rawfile[,2]
dataTraining <− t(rawfile[,-c(1,2)])
ProteinNames <− read.delim(filnamnTraining,header=FALSE)
ProteinNames <− as.character(as.matrix(ProteinNames)[1,])
ProteinNames <− ProteinNames[-(1:2)]
listaBoolean <− is.element(ProteinNames, lista)
rownames(dataTraining) <− ProteinNames
colnames(dataTraining) <− samplenames
logdataTraining <− dataTraining
logdataTraining <− logdataTraining[listaBoolean,]
rawfile <− read.delim(filnamnTest)
samplenames <− as.character(rawfile[,1] )
groupsTest <− rawfile[,2]
dataTest <− t(rawfile[,-c(1,2)])
ProteinNames <− read.delim(filnamnTest,header=FALSE)
ProteinNames <− as.character(as.matrix(ProteinNames)[1,)}
ProteinNames <− ProteinNames[-(1:2)]
rownames(dataTest) <− ProteinNames
colnames(dataTest) <− samplenames
logdataTest<−dataTest
logdataTest <− logdataTest[listaBoolean,]
svmfacTraining<− factor(rep(‘rest’,ncol(logdataTraining)),levels=c(group1,
group2, ‘rest’))
subset1Training<− is.element(groupsTraining ,
strsplit(group1,“,”)[[1]])
subset2Training<− is.element(groupsTraining ,
strsplit(group2,“,”)[[1]])
svmfacTraining[subset1Training] <− group1
svmfacTraining[subset2Training] <− group2
facTraining <−factor(as.character(svmfacTraining
[subset1Training|subset2Training]),levels=c(group1,group2))
svmfacTest<− factor(rep(‘rest’,ncol(logdataTest)),levels=c(group1, group2,
‘rest’))
subset1Test<− is.element(groupsTest , strsplit(group1,“,”)[[1]])
subset2Test<− is.element(groupsTest , strsplit(group2,“,”)[[1]])
svmfacTest[subsetlTest] <− group1
svmfacTest[subset2Test] <− group2
facTest <−factor(as.character(svmfacTest
[subset1Test|subset2Test]),levels=c(group1,group2))
  n1 <− sum(facTest ==levels(facTest )[1])
  n2 <− sum(facTest ==levels(facTest )[2])
  nsamples <− n1+n2
SampleInformation <− paste(levels(facTest )[1],“ “,n1,” , “, levels(facTest
)[2],” “,n2,sep=””)
svmtrain <− svm(t(logdataTraining) , facTraining , kernel=“linear” )
pred<−predict(svmtrain , t(logdataTest) , decision.values=TRUE)
res<−attr(pred, “decision.values”)
names <− colnames(logdataTest, do.NULL=FALSE)
orden <− order(res , decreasing=TRUE)
Samples <− data.frame(names[orden],res[orden],facTest[orden])
ROCdata <− myROC(res,facTest)
SenSpe <− SensitivitySpecificity(res,facTest )
ROCplot(list(SampleInformation=SampleInformation,ROCarea=ROCdata[1],p
.value=ROCdata[2],SenSpe <− SenSpe,samples=Samples),
sensspecnumber=4 )
#rows in blue are needed only for ROC evaluation.
#to assess an unknown sample, print res.
#(vector with prediction values)
```

1. An in vitro method for identifying agents capable of inducing sensitization of mammalian skin comprising or consisting of the steps of:
a) exposing a population of dendritic cells or a population of dendritic-like cells to a test agent; and
b) measuring in the cells the expression of one or more biomarker(s) selected from the group defined in Table 3;
wherein the expression in the cells of the one or more biomarkers measured in step (b) is indicative of the sensitizing effect of the sample to be tested.

2. The method according to claim 1 further comprising exposing a separate population of the dendritic cells or dendritic-like cells to a negative control agent that does not sensitize human skin and measuring in the cells the expression of the one or more biomarker(s) measured in step (b).

3. The method according to claim 1 further comprising exposing a separate population of the dendritic cells or dendritic-like cells to a positive control agent that sensitizes human skin and measuring in the cells the expression of the one or more biomarker(s) measured in step (b).

4. The method according to claim 1 wherein step (b) comprises measuring the expression of at least one biomarker selected from the group consisting of:
i) taste receptor, type 2, member 5 (TAS2R5),
ii) keratinocyte growth factor-like protein 1/2/hypothetical protein FLJ20444 (KGFLP1/2/FLJ20444),
iii) transmembrane anterior posterior transformation 1 (TAPT1),
iv) sprouty homolog 2 (SPRY2),
v) fatty acid synthase (FASN),
vi) B-cell CLL/lymphoma 7A (BCL7A),
vii) solute carrier family 25, member 32 (SLC25A32),
viii) ferritin, heavy polypeptide pseudogene 1 (FTHP1),
ix) ATPase, H+ transporting, lysosomal 50/57 kDa, V1 subunit H (ATP6V1H),
x) squalene epoxidase (SQLE), and
xi) histone cluster 1, Hie (HIST1H1E).

5-15. (canceled)

16. The method according to claim 1 wherein step (b) comprises or consists of measuring the expression of at least 2 biomarkers from Table 3A, for example, at least 3, 4, 5, 6, 7, 8, 9, 10 or 11 biomarkers from Table 3A.

17. The method according to claim 1 wherein step (b) comprises or consists of measuring the expression of at least 2 biomarkers from Table 3B, for example, at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, or at least 189 biomarkers from Table 3B.

18-20. (canceled)

21. The method according to claim 1 wherein step (b) comprises measuring the expression of a nucleic acid molecule encoding the one or more biomarker(s).

22. (canceled)

23. (canceled)

24. The method according to claim 21 wherein measuring the expression of the one or more biomarker(s) in step (b) is performed using a method selected from the group consisting of Southern hybridisation, Northern hybridisation, polymerase chain reaction (PCR), reverse transcriptase PCR (RT-PCR), quantitative real-time PCR (qRT-PCR), nanoarray, microarray, macroarray, autoradiography and in situ hybridisation.

25. (canceled)

26. The method according to claim 1 wherein measuring the expression of the one or more biomarker(s) in step (b) is performed using one or more binding moieties, each capable of binding selectively to a nucleic acid molecule encoding one of the biomarkers identified in Table 3.

27-36. (canceled)

37. The method according to claim 1 wherein step (b) comprises measuring the expression of the protein of the one or more biomarker(s).

38. The method according to claim 37 wherein measuring the expression of the one or more biomarker(s) in step (b) is performed using one or more binding moieties each capable of binding selectively to one of the biomarkers identified in Table 3.

39-45. (canceled)

46. The method according to claim 1 wherein step (b) is performed using an array.

47-55. (canceled)

56. The method according to claim 1 for identifying agents capable of inducing allergic contact dermatitis (ACD).

57. The method according to claim 1 wherein the population of dendritic cells or population of dendritic-like cells is a population of dendritic-like cells.

58-60. (canceled)

61. The method according to claim 57 wherein the myeloid leukaemia-derived cells are selected from the group consisting of KG-1, THP-1, U 937, HL-60, Monomac-6, AML-193 and MUTZ-3.

62. (canceled)

63. The method according to claim 1 wherein the control non-sensitizing agent(s) provided in step (e) is selected from the group consisting of 1-Butanol, 4-Aminobenzoic acid, Benzaldehyde, Chlorobenzene, Diethyl phthalate, Dimethyl formamide, Ethyl vanillin, Glycerol, Isopropanol, Lactic acid, Methyl salicylate, Octanoic acid, Propylene glycol, Phenol, p ydroxybenzoic acid, Potassium permanganate, Salicylic acid, Sodium dodecyl sulphate, Tween 80 and Zinc sulphate.

64. (canceled)

65. The method according to claim 1 wherein the control sensitizing agent(s) provided in step (i) is selected from the group consisting of 2,4-Dinitrochlorobenzene, Oxazolone, Potassium dichromate, Kathon CH (MC/MCI), Formaldehyde, 2-Aminophenol, 2-nitro-1,4-Phenylendiamine, p Phenylendiamine, Hexylcinnamic aldehyde, 2-Hydroxyethyl acrylate, 2 Mercaptobenzothiazole, Glyoxal, Cinnamaldehyde, Isoeugenol, Ethylendiamine, Resorcinol, Cinnamic alcohol, Eugenol, Penicillin G or Geraniol.

66-68. (canceled)

69. An array for use in a method defined in claim 1, the array comprising one or more binding moieties each capable of binding selectively to a nucleic acid molecule encoding one of the biomarkers identified in Table 3.

70-73. (canceled)

74. Use of two or more biomarkers selected from the group defined in Table 3A or Table 3B in combination for identifying hypersensitivity response sensitising agents.

75. (canceled)

76. An analytical kit for use in a method defined in claim 1 comprising:
A) an array for use in a method defined in claim 1; and, optionally,
B) instructions for performing the method as defined in claim 1.

77-81. (canceled)
